(19) United States
(12) Patent Application Publication
(10) Pub. No.: US 2011/0008886 A1
(43) Pub. Date: Jan. 13, 2011

(54) METHODS AND MATERIALS FOR ISOLATING ISOGENIC ISLET CELLS

(75) Inventors: Bernhard J. Hering, Minnetonka, MN (US); Shinichi Matsumoto, Kita-ku Kyoto (JP)

Correspondence Address: FISH & RICHARDSON P.C. (TC), PO BOX 1022, MINNEAPOLIS, MN, 55440-1022, US

(73) Assignee: REGENTS OF THE UNIVERSITY OF MINNESOTA, Saint Paul, MN (US)

(21) Appl. No.: 12/776,648

(22) Filed: May 10, 2010

Related U.S. Application Data
(62) Division of application No. 11/056,897, filed on Feb. 11, 2005, now abandoned.

(51) Int. Cl. C12N 5/071; C12N 5/00

(52) U.S. Cl. 435/366; 435/404

(57) ABSTRACT

Compositions and methods for isolating and purifying islet cells are described. Islets obtained using such compositions and methods can be transplanted into diabetic patients.

### CROSS-REFERENCE TO RELATED APPLICATIONS

This application is a divisional of U.S. application Ser. No. 11/056,897, filed on Feb. 11, 2005.

### TECHNICAL FIELD

This invention relates to methods and materials for isolating islet cells, and more particularly to methods and materials for isolating islet cells from a single donor.

### BACKGROUND

Type 1 diabetes mellitus continues to be a therapeutic challenge. Failure to prevent hypoglycemia and hyperglycemia results in acute and chronic complications, leading to poor quality of life, premature death, and considerable health care costs in 30% to 50% of diabetic patients. Establishing safe, effective ways to achieve and maintain normoglycemia would have substantial implications for the well-being of individuals with diabetes. Intensive insulin therapy has been shown to reduce the risk of chronic complications in patients who achieve near-normalization of glycemia. Such therapy, however, is labor-intensive, difficult to implement for many patients, and limited by the increased frequency of severe hypoglycemia. Currently, the only way to restore and sustain normoglycemia without the associated risk of hypoglycemia is by replacing the patient's islets of Langerhans, either by transplanting a vascularized pancreas or, much less invasively, by infusing isolated islets from multiple donors.

### SUMMARY

The invention is based on the discovery of methods and materials for isolating and purifying islets from single donors such that normoglycemia can be sustained and insulin independence can be achieved after transplanting the cells in patients with diabetes (e.g., type 1 diabetes). To isolate islets in high yield from single donors, ischemic injury of islets is limited during pancreas storage and islet-toxic reagents are avoided during islet processing. Furthermore, isolated islets are cultured before transplantation to allow pretransplant initiation of immunosuppression. Such methods and materials reduce the risks and costs of islet transplants and thereby increase the availability of islet transplants to a greater patient population.

In one aspect, the invention features a composition that includes 16.00 to 20.00 g/L raffinose; 4.00 to 6.00 g/L histidine; 4.00 to 5.00 g/L sodium hydroxide; 30.00 to 40.00 g/L lactobionic acid; 0.30 to 0.50 g/L potassium hydroxide; 0.05 to 0.10 g/L calcium chloride; 1.00 to 1.50 g/L magnesium sulfate; 3.00 to 4.00 g/L sodium phosphate monobasic; and 19.00 to 21.00 g/L pentastarch. The composition further can include 8.00 to 12.00 U/mL heparin and 8.00 to 12.00 μg/mL insulin. In some embodiments, the composition also includes iodixanol. The composition also can include a population of human pancreatic islets (e.g., an isogenic population of human pancreatic islets). The composition can be substantially free of pancreatic cells non-isogenic to the human pancreatic islets.

In another aspect, the invention features a composition that includes 5.00 to 6.00 g/L mannitol; 0.50 to 0.70 g/L sodium hydroxide; 5.00 to 7.00 g/L sodium chloride; 0.25 to 0.40 g/L potassium hydroxide; 0.05 to 0.15 g/L calcium chloride; 0.15 to 0.25 g/L magnesium sulfate; and 3.00 to 4.00 g/L sodium phosphate monobasic. The composition can include 8.00 to 12.00 U/mL heparin and/or 1,000 to 3600 Wunsch units of collagenase. The composition also can include a trypsin inhibitor (e.g., 4-(2-aminoethyl)-benzenesulfonyl fluoride hydrochloride, TLCK (1-Chloro-3-tosylamido-7-amino-2-heptanone HCl), or trypsin inhibitor from soybean). In some embodiments, the composition also includes a population of human pancreatic islets (e.g., an isogenic population of human pancreatic islets). The composition can be substantially free of pancreatic cells non-isogenic to the human pancreatic islets.

The invention also features a composition including 16.00 to 20.00 g/L raffinose; 4.00 to 6.00 g/L histidine; 4.00 to 5.00 g/L sodium hydroxide; 30.00 to 40.00 g/L lactobionic acid; 0.30 to 0.50 g/L potassium hydroxide; 0.05 to 0.10 g/L calcium chloride; 1.00 to 1.50 g/L magnesium sulfate; 3.00 to 4.00 g/L sodium phosphate monobasic; 15.00 to 25.00 g/L pentastarch; and 200 to 300 ml/L iodixanol. In some embodiments, the composition also includes a population of human pancreatic islets (e.g., an isogenic population of human pancreatic islets). The composition can be substantially free of pancreatic cells non-isogenic to the human pancreatic islets.

In another aspect, the invention features a preparation of isolated, isogenic human pancreatic islets, wherein the preparation contains at least 2.2×10⁵ islet equivalents (IE) (e.g., at least 2.7×10⁵ IE or 3.5×10⁵ IE). The preparation can exhibit an oxygen consumption rate of greater than 75 nmol/min/mg DNA (e.g., greater than 230 nmol/min/mg DNA). The preparation can exhibit an ATP/DNA ratio of at least 110 pmol ATP/μg DNA. The islets can include α, β, γ, PP, acinar, and ductal cells. The preparation further can include a cryopreservative (e.g., dimethylsulfoxide).

In yet another aspect, the invention features a preparation of isolated, isogenic human pancreatic islets for transplantation into a human patient in need thereof, where the preparation is characterized, prior to transplant, as having at least a 60% probability of constituting a successful transplant.

The invention also features a collection of at least five cryopreserved preparations of isolated, isogenic human pancreatic islets, wherein at least 60% of the preparations, when transplanted individually, are capable of constituting a successful pancreatic islet transplant for a patient in need thereof.

A method of characterizing the transplant potency of a preparation of isolated, isogenic human pancreatic islets also is featured. The method includes assaying the preparation for the ATP/DNA ratio, the oxygen consumption rate (OCR)/DNA ratio, and beta cell number; and characterizing the transplant potency on the basis of the assay results.

In yet another aspect, the invention features a chemically defined culture medium that includes insulin, zinc sulfate, selenium, and transferrin, wherein the medium is effective for maintaining viability of human pancreatic islets under culture conditions. The culture medium further can include sodium pyruvate, HEPES (N-[2-Hydroxyethyl]piperazine-N′[2-ethanesulfonic acid]), and human serum albumin (HSA). In some embodiments, the culture medium further includes 8.00 to 12.00 U/mL heparin. The culture medium also can include a population of human pancreatic islets (e.g., an isogenic population of human pancreatic islets). The culture medium can be substantially free of pancreatic cells non-isogenic to the human pancreatic islets.

The invention also features a composition that includes 8.00 to 10.00 g/L mannitol; 3.00 to 6.00 g/L L-histidine; 18.00 to 21.00 g/L gluconic acid; 0.50 to 2.00 g/L potassium hydroxide; 0.01 to 0.05 g/L calcium chloride; 0.50 to 2.00 g/L magnesium sulfate; 0.40 to 0.70 g/L nicotinamide; 0.30 to 0.70 g/L pyruvate; and 1.50 to 3.50 g/L potassium phosphate monobasic. In some embodiments, the composition also includes a population of human pancreatic islets (e.g., an isogenic population of human pancreatic islets). The composition can be substantially free of pancreatic cells non-isogenic to the human pancreatic islets.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a schematic of the circulation system for digestion of the pancreas. In FIG. 1B, the schematic indicates the flow direction from the 1 L beaker to the chamber and from the chamber to the 250 mL conical tube. In FIG. 1C, the schematic indicates the flow direction during Phase I (from 250 mL conical tube to chamber and from chamber to 250 mL conical tube). FIG. 1D is a schematic of the circulation system at the switch from Phase Ito Phase II, with flow directions provided from the 250 mL conical tube to the chamber and from the chamber to the collection flask. FIG. 1E is a schematic of the circulation system during Phase II, with flow directions from the 1 L beaker to the chamber and the chamber to the collecting flask.

FIG. 2 is a table describing the recipient characteristics, drug exposure, and portal vein access route for 8 islet recipients.

FIG. 3 is a table describing the autoantibody levels relative to day of transplant of 8 islet recipients.

FIG. 4 contains two graphs of the pre hOKT3γ1 (Ala-Ala) and peak post hOKT3γ1 (Ala-Ala) serum IL-2 (left panel) and IL-10 levels (right panel) in individual subjects. Serum was collected prior to the first hOKT3γ1 (Ala-Ala) dose and at 3 hours post-dose on days 1-7, and assayed by ELISA for IL-2 and IL-10 levels. Subject #1 is represented by the solid black line; subject #2 by the solid black rectangles; #3 by the horizontal striped line; #4 by the solid gray line; #5 by the solid gray squares; and #6 by the dashed black line.

FIG. 5 contains graphs representing the number of circulating CD2⁺ (left upper panel), CD4⁺ (left middle panel), and CD8⁺ (left lower panel) T cells as analyzed by flow cytometry; as well as the CD4⁺:CD8⁺ T cell ratio (right upper panel); the percentage of CD4⁺CD25⁺ of CD4⁺ T cells (right middle panel); and percentage of CD4⁺CD69⁺ of CD4⁺ T cells (right lower panel). Subject #1 is represented by the solid black line; subject #2 by the solid black rectangles; #3 by the horizontal striped line; #4 by the solid gray line; #5 by the solid gray squares; and #6 by the dashed black line.

FIG. 6 is a graph of the percent circulating CD25⁺ T cells within the CD4⁺ subset in healthy controls, type 1 diabetic individuals, and type 1 diabetic islet transplant recipients after treatment with hOKT3γ1 (Ala-Ala), rapamycin, and reduced-dose tacrolimus. Peripheral blood from type 1 diabetic islet allograft recipients who received hOKT3γ1 (Ala-Ala) induction therapy and sirolimus combined with tacrolimus maintenance immunosuppression (closed circles, ), non-transplanted long-term type 1 diabetics (closed triangles, ▴), and healthy nondiabetic controls (closed squares, ▪) was stained with PE conjugated anti-CD4-PE, FITC-conjugated anti-CD25, and PerCP-conjugated anti-CD45. Stained cells were analyzed by flow cytometry. Lymphocytes were gated by forward, side scatter, and CD45-PerCP intensity, then the fraction of CD25+ cells within the CD4⁺ T cell subset was determined. Peak percentages are shown for type 1 diabetic islet recipients.

FIG. 7 is a bar graph of the percent reduction in proliferative response of CD4⁺CD25⁻ T cells to donor (dark bars) and third party cells (light bars) in the presence of CD4⁺CD25⁺ T cells at a ratio of 1:1. Peripheral blood CD4⁺CD25⁺ and CD4⁺CD25⁻ T cell subsets were isolated by sorting on a flow cytometer (patients 1,2,3,5) or by separation on an anti-CD25 magnetic bead column (patient 6). CD4⁺CD25⁻ T cells cultured alone or with added equal numbers of CD4⁺CD25⁺ T cells were stimulated with irradiated allogeneic or third-party splenocytes. Proliferative events by CD4⁺CD25⁻-T cells in stimulated cultures were identified by decreased CFSE fluorescence compared to fluorescence of cultures with CFSE-labeled, unstimulated CD4⁺CD25⁻-T cells. In some experiments in which the cultured cells were also stained with anti-CD25-PE on the day of culture harvest, the proliferating cells were found to be CD25⁺.

### DETAILED DESCRIPTION

In general, the invention provides methods and materials for isolating and purifying islet cells from donors, and in particular, for isolating and purifying isogenic islets, i.e., islets from a single donor. The quality of islets obtained using the methods and materials described herein allows a lower number of isogenic islets to be transplanted into a patient with any type of diabetes, including, for example, type 1 diabetes, type 2 diabetes, and surgical diabetes. This results in an increase in the number of donor pancreases from which islet cells can be isolated and increases the availability of islet transplants to a greater patient population.

### Procuring and Preserving Donor Pancreases

Pancreases can be obtained from male or female donors in accordance with federal regulations (e.g., 21 C.F.R. §1270) and techniques developed for combined liver and pancreaticoduodenal procurement (Marsh et al., Surg. Gynecol. Obstet. 1989; 168:254-258). Donors typically range in age from 15 to 50 years old. General exclusion criteria include, for example, systemic bacterial infections, viruses such as human immunodeficiency virus (HIV), human T-cell lymphotrophic virus (HTLV), hepatitis B virus, or hepatitis C virus (HCV), a history of diabetes, extracranial tumors, and risk factors for AIDS.

Donor pancreases can be preserved using the two-layer pancreas preservation method, which improves pancreatic tissue adenosine triphosphate (ATP) content, increases the yield of islets isolated from a stored pancreas, allows use of marginal donor pancreases for islet isolation and transplantation, improves the islet isolation success rate, and preserves the integrity of the isolated islets (e.g., such that isolated islets can reverse diabetes). In general, cold University of Wisconsin (UW) Solution (ViaSpan®, DuPont Pharma, Wilmington, Del.) (see U.S. Pat. Nos. 4,798,824 and 4,879,283) or modified UW solution can be poured on top of an equal volume of cold perfluorodecalin (FluoroMed, L. P., Round Rock, Tex.). Typically, the two-layer preservation method is performed in an organ shipping container, which has, for example, a removable lid with a stainless steel mesh plate attached thereto, and inlet and outlet ports. See, for example, the organ shipping container of U.S. Pat. No. 6,490,880.

Two layers are formed after adding ViaSpan® or modified-UW solution to the perfluorodecalin as the specific gravity of perfluorodecalin is greater than ViaSpan® and modified-UW solution. Modified UW solution includes 0.35 to 0.45 g/L potassium hydroxide, 3.00 to 4.00 g/L monosodium phosphate monohydrate, 0.05 to 1.00 g/L calcium chloride dihydrate, 1.10 to 1.30 g/L magnesium sulfate heptahydrate, 33.00 to 38.00 g/L lactobionic acid, 4.00 to 5.00 g/L L-histidine, 15.00 to 20.00 g/L raffinose, 4.00 to 5.00 g/L sodium hydroxide, 15.00 to 25.00 g/L penta starch, 1.00 to 1.50 g/L adenosine, and 0.75 to 1.50 g/L glutathione. In particular, the modified UW solution can include 0.39 g/L potassium hydroxide, 3.45 g/L monosodium phosphate monohydrate, 0.074 g/L calcium chloride dihydrate, 1.23 g/L magnesium sulfate heptahydrate, 35.83 g/L lactobionic acid, 4.66 g/L L-histidine, 17.84 g/L raffinose, 4.60 g/L sodium hydroxide, 20.00 g/L penta starch, 1.34 g/L adenosine, and 0.92 g/L glutathione. Typically, the perfluorodecalin is oxygenated for 30-70 minutes (e.g., 40-60 minutes). For example, medical grade oxygen can be filtered through a 0.2 mm filter (Gelman Sciences, Ann Arbor, Mich.) and the inlet port of the shipping container at a rate of 2.5 L/min. Preferably, the cold storage time of the donor pancreas is less than 12 hours (e.g., less than 10, 8, 6, 4, or 2 hours).

### Isolating Islets Using the Automated Method for Pancreatic Tissue Dissociation

Upon receipt of a donor pancreas, integrity of the shipping container can be verified by visual inspection. The pancreas can be removed and rinsed with cold transport solution containing 8.00 to 10.00 g/L mannitol, 3.00 to 6.00 g/L L-histidine, 18.00 to 21.00 g/L gluconic acid, 0.50 to 2.00 g/L potassium hydroxide, 0.01 to 0.05 g/L calcium chloride, 0.50 to 2.00 g/L magnesium sulfate, 0.40 to 0.80 g/L nicotinamide, 0.30 to 0.70 g/L pyruvate, and 1.50 to 3.50 g/L potassium phosphate monobasic. For example cold transport solution can include 8.50 to 9.50 g/L (e.g., 9.11 g/L) D-mannitol, 4.00 to 5.00 g/L (e.g., 4.67 g/L) L-histidine, 18.50 to 20.50 g/L (e.g., 19.63 g/L) D-gluconic acid sodium salt, 0.80 to 1.40 g/L (e.g., 1.12 g/L) potassium hydroxide, 0.025 to 0.045 g/L (e.g., 0.037 g/L) calcium chloride dihydrate, 1.00 to 1.50 g/L (e.g., 1.23 g/L) magnesium sulfate heptahydrate, 0.55 to 0.65 g/L (e.g., 0.61 g/L) nicotinamide, 0.50 to 0.60 g/L (e.g., 0.55 g/L) sodium pyruvate, and 2.50 to 3.25 g/L (e.g., 2.72 g/L) potassium phosphate monobasic.

Islets can be isolated from the donor pancreas using an automated method of pancreatic tissue dissociation. See, for example, Ricordi et al., Diabetes 1988; 37:413-420. This method includes the general steps of 1) dissection; 2) distension; 3) dissociation; and 4) collection.

Dissection of the pancreas can include removing extraneous fat (while retaining some fat to minimize leaking during distension), and non-pancreatic tissue. Typically, about 80% to about 95% of the fat is removed. The dissected pancreas can be incubated in a topical antibiotic solution containing, for example, gentamicin (Elkins-Sinn, Inc.), Cefazolin (SmithKline Beecham Pharmaceutical), and amphotericin-B (Apothecon®) in cold transport solution, then can be serially rinsed in phenol red-free Hanks' Balanced Salt Solution (Mediatech, Inc., Herndon, Va.). The pancreas can be divided at the neck into the ‘body and tail’ and ‘head’ and the following steps performed on each part. In general, the pancreatic duct can be cannulated with an angiocatheter (16-20 gauge) and the pancreas perfused under controlled conditions, including an initial pressure of 80 mmHg followed by an increase in pressure to 180 mmHg for the remainder of the distension procedure. Phase I solution can be used to perfuse the pancreas. Phase I solution includes 5.00 to 6.00 g/L mannitol, 0.50 to 0.70 g/L sodium hydroxide, 5.00 to 7.00 g/L sodium chloride, 0.25 to 0.40 g/L potassium hydroxide, 0.05 to 0.15 g/L calcium chloride, 0.15 to 0.25 g/L magnesium sulfate, and 3.00 to 4.00 g/L sodium phosphate monobasic. For example, Phase I solution can include 5.47 g/L D-mannitol, 0.60 g/L sodium hydroxide, 6.14 g/L sodium chloride, 0.33 g/L potassium hydroxide, 0.11 g/L calcium chloride dihydrate, 0.20 g/L magnesium sulfate heptahydrate, and 3.45 g/L sodium phosphate monobasic.

Typically, the Phase I solution contains 1,000 to 3,600 Wunsch units (collagenase activity) or 28,000 to 128,500 caseinase units (proteolytic activity) of collagenase. For example, the Phase I solution can include 1500 to 3000 (e.g., 1,562 to 2,954 or 2,082 to 2,363) Wunsch units, or 42,000 to 108,000 (e.g., 42,328 to 107,064 or 56,437 to 85,651) caseinase units of collagenase. A suitable collagenase includes Liberase™HI (Roche Molecular Biochemicals, Indianapolis, Ind.), which has been specifically formulated for human islet isolation procedures. See, Linetsky et al., Diabetes 1997; 46:1120-1123. Preferably, powdered Liberase™HI is reconstituted at least 20 minutes before, but less than 2 hours before, addition to the Phase I solution.

The Phase I solution also can include a protease inhibitor (e.g., a trypsin inhibitor such as 4-(2-aminoethyl)-benzenesulfonyl fluoride hydrochloride (Pefabloc® SC PLUS), TLCK (1-Chloro-3-tosylamido-7-amino-2-heptanone HCl), or trypsin inhibitor from soybean). For example, the Phase I solution can include 0.05 to 0.15 mg/mL of Pefabloc® SC PLUS, which specifically inhibits endogenous proteases and decreases auto-digestion. The Phase I solution also can include 8 to 12 units/mL of heparin (e.g., Monoparin®, Accurate Chemical and Scientific Corporation). For example, the Phase I solution can include 10 units/mL of heparin.

In some embodiments, the Phase I solution contains 1,000 to 3,600 Wunsch units of collagenase, 0.05 to 0.15 mg/mL of a trypsin inhibitor, and 10 units/mL of heparin. After a sufficient period of time of cold perfusion, e.g., 8-20 minutes, the distended pancreas can be further trimmed of remaining capsule and placed into a dissociation chamber (e.g., a sterile stainless steel chamber (Wahoff et al., Ann. Surg. 1995; 222:562-579), also known as a Ricordi chamber). Collagenase that “leaked” from the distended pancreas can be added to the chamber.

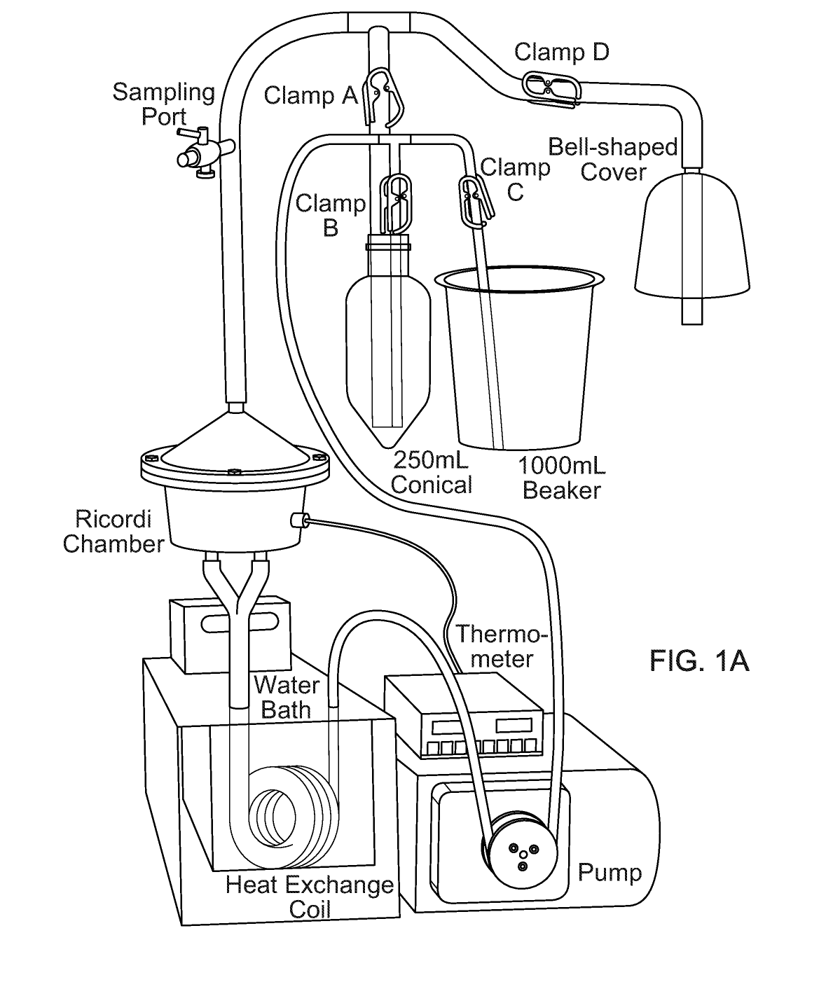

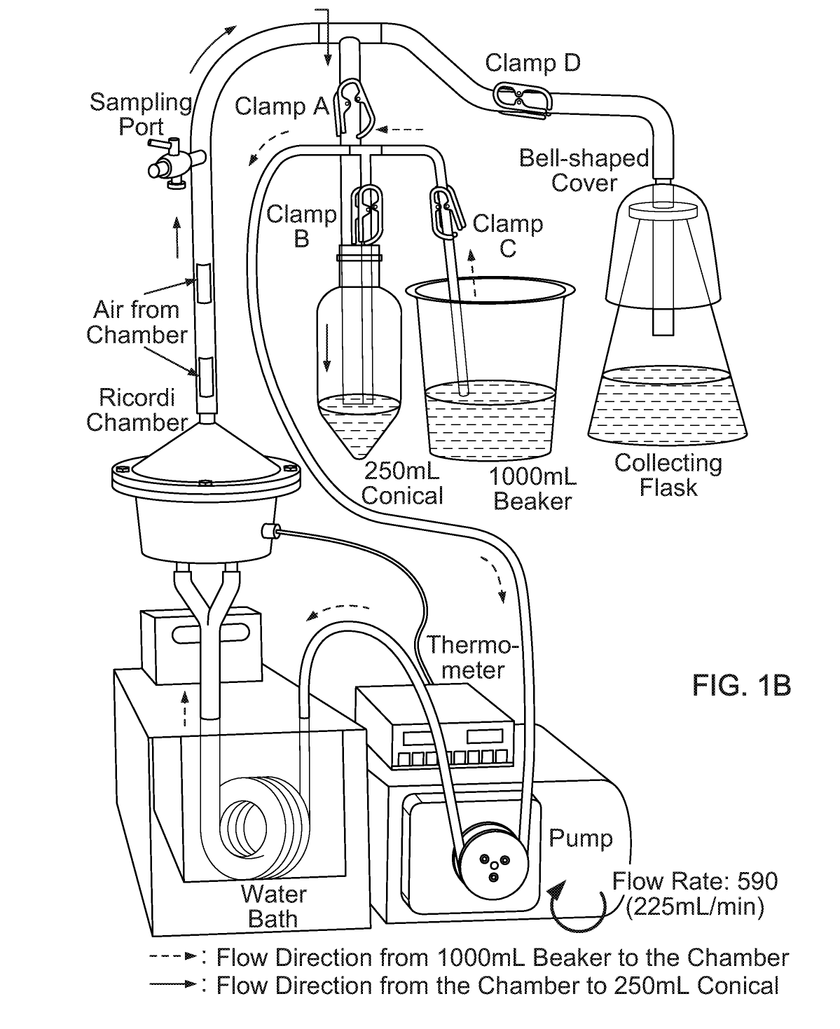

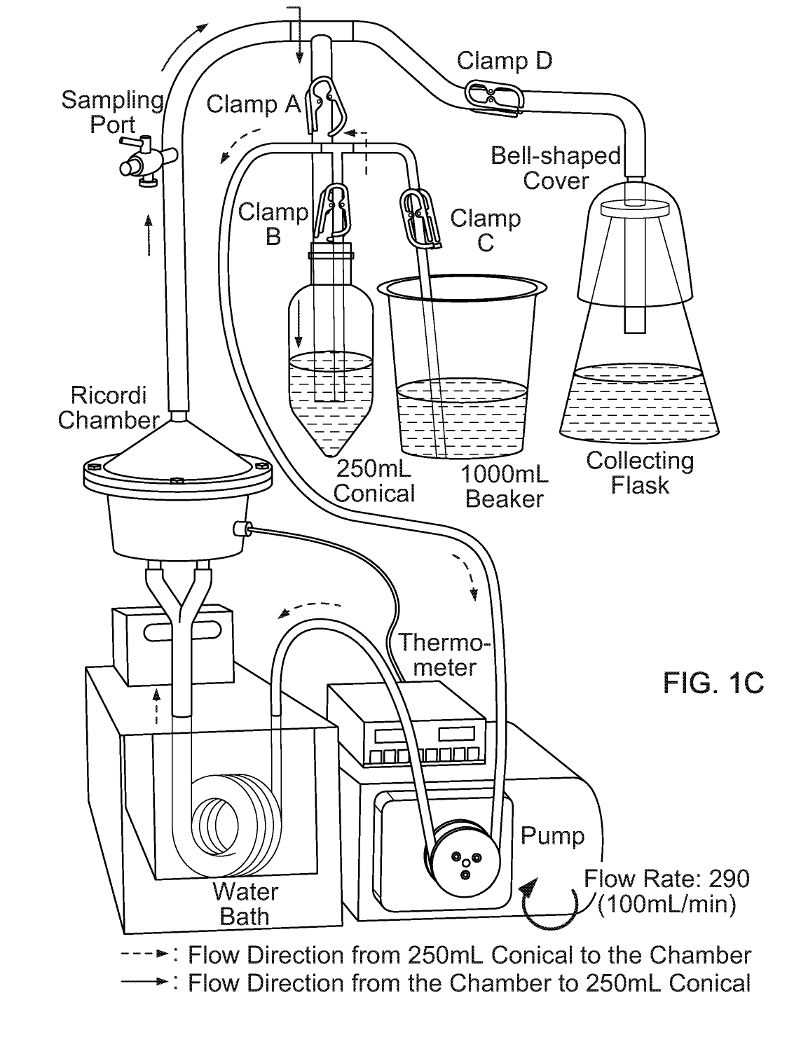

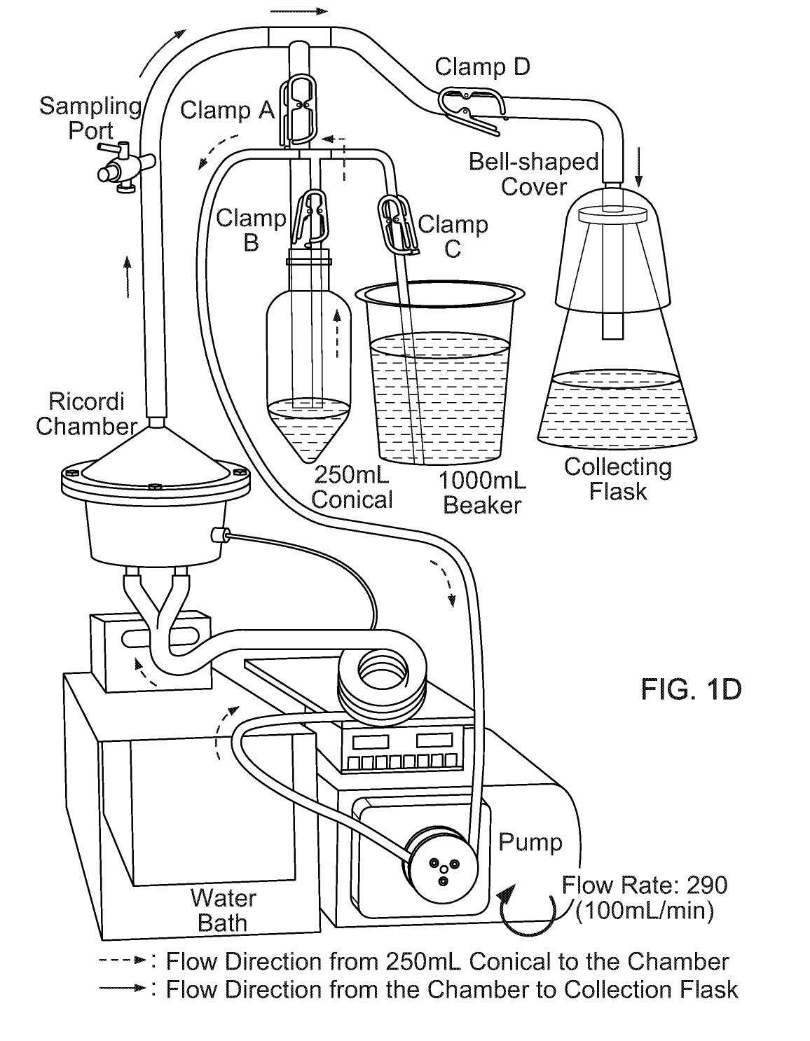

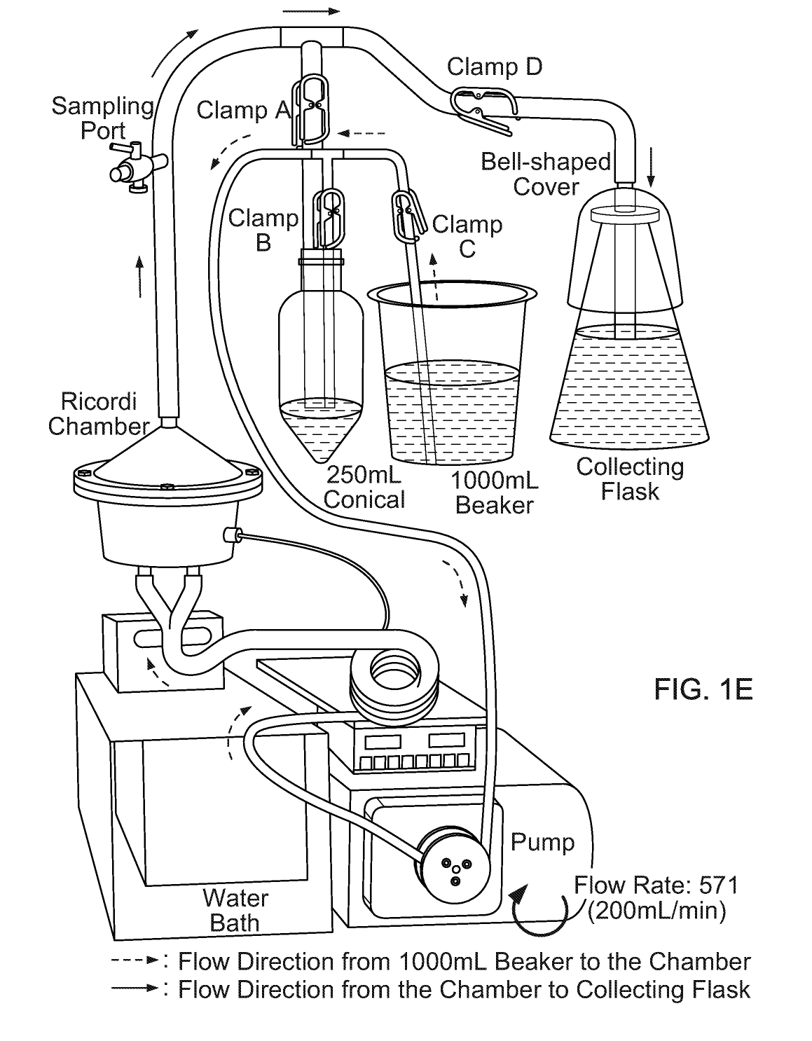

Typically, the Ricordi chamber is in a circulation system that includes a heat exchange coil (e.g., a stainless steel coil), a pump, a temperature monitor and sensor, a loading flask, a fluid collection flask, a sample collecting flask, and tubes for fluidly connecting components. Flow direction can be controlled using, for example, valves or clamps. The heat exchange coil can be placed in a water bath. FIG. 1A shows one embodiment of a circulation system that contains a Ricordi chamber, a stainless steel coil for heat exchange, six (6) tubes with small diameter (Master Flex tubing, size 16), four (4) tubes with large diameter (Master Flex tubing, size 17), steel 3-way stopcock for sampling, four (4) plastic clamps, 250 mL conical tube, tri-pour graduated disposable beaker, 1000 mL, bell-shaped plastic cover, two (2) T-connectors, (1) T-connector with luer lock port, and one (1) Y-connector, 18 inch steel ring stand with two arms, Ismatec pump, Mon-a-therm temperature monitor and sensor, and water bath.

The system can be filled with Phase I solution and air evacuated to begin the digestion phase. In particular, Phase I solution can be allowed to flow from the loading flask (e.g., the 1 L beaker in FIG. 1A) through the pump, heat exchange coil, and Ricordi chamber to the fluid collecting flask (e.g., the 250 mL conical tube in FIG. 1A). After 10% to 30% of the volume of Phase I solution reaches the fluid collecting flask, the flow of the system can be adjusted such that the Phase I solution is recirculated through the system, i.e., the Phase I solution flows from the fluid collecting flask to the chamber and from the chamber to the fluid collecting flask. The chamber can be agitated while the fluid is being recirculated to aid tissue dissociation. Temperature of the fluid can be maintained at 25° C. to 37° C.

The collection phase can begin once there is an increase in the amount of tissue liberated from the chamber, most or all of the islets are free of the surrounding acinar tissue, intact islets are observed, and the acinar tissue becomes finer (small cell clusters). Diphenylthiocarbazone (DTZ) staining can be used to distinguish islets from non-islet tissue. See, Latif et al., Transplantation 1988; 45:827-830. DTZ selectively binds to the zinc-insulin complex in islet beta cell granules, and results in a red staining of the islets. DTZ staining provides a rapid means for discrimination of islet from acinar tissue, and the positive reaction indicates that insulin-containing beta cells are present.

During the collection phase, temperature of the system can be reduced to about 10° C. to about 30° C. Fluid in the fluid collecting flask can be allowed to flow through the pump and heat exchange coil into the Ricordi chamber, and Phase II solution (RPMI 1640, catalog #99-595-CM, Mediatech, Inc., Herndon, Va.) can be added to a loading flask. The Phase II solution can be pumped through the circulation system to dilute the collagenase and to wash the tissue. Digested material can be collected in flasks containing Phase II solution and human serum albumin (HSA), and the collected material washed two to five times using cold storage solution. Cold storage solution can include 16.00 to 20.00 g/L raffinose, 4.00 to 6.00 g/L histidine, 4.00 to 5.00 g/L sodium hydroxide, 30.00 to 40.00 g/L lactobionic acid, 0.30 to 0.50 g/L potassium hydroxide, 0.05 to 0.10 g/L calcium chloride, 1.00 to 1.50 g/L magnesium sulfate, 3.00 to 4.00 g/L sodium phosphate monobasic, 19.00 to 21.00 g/L pentastarch, 8.00 to 12.00 U/mL heparin, and 8.00 to 12.00 μg/mL insulin. For example, cold storage solution can include 17.84 g/L D (+) raffinose, 4.66 g/L L-histidine, 4.60 g/L sodium hydroxide, 35.83 g/L lactobionic acid, 0.39 g/L potassium hydroxide, 0.39 g/L calcium chloride dihydrate, 1.23 g/L magnesium sulfate heptahydrate, 3.45 g/L sodium phosphate monobasic, 2% penta starch, 10 U/mL heparin, and 10 μg/mL insulin.

Cold storage solution can be made by combining H-Phase II solution (80% by volume) with 10% penta starch (i.e., 100 g/L) (20% by volume), and adding 8.00 to 12.00 U/mL heparin, and 8.00 to 12.00 μg/mL insulin. H-Phase II solution can include 16.00 to 20.00 g/L raffinose, 4.00 to 6.00 g/L histidine, 4.00 to 5.00 g/L sodium hydroxide, 30.00 to 40.00 g/L lactobionic acid, 0.30 to 0.50 g/L potassium hydroxide, 0.05 to 0.10 g/L calcium chloride, 1.00 to 1.50 g/L magnesium sulfate, and 3.00 to 4.00 g/L sodium phosphate monobasic. The pH of H-Phase II solution can be adjusted to a pH of 7.3-7.5 using hydrochloric acid or sodium hydroxide. Density of H-Phase II solution typically is 1.063±0.003. For example, H-Phase II solution can include 17.84 g/L D (+) raffinose, 4.66 g/L L-histidine, 4.60 g/L sodium hydroxide, 35.83 g/L lactobionic acid, 0.39 g/L potassium hydroxide, 0.39 g/L calcium chloride dihydrate, 1.23 g/L magnesium sulfate heptahydrate, and 3.45 g/L sodium phosphate monobasic.

The washed tissue can be resuspended in capping layer solution and HSA (e.g., 25% HSA). Capping layer solution can include 16.00 to 20.00 g/L raffinose; 4.00 to 6.00 g/L histidine; 4.00 to 5.00 g/L sodium hydroxide; 30.00 to 40.00 g/L lactobionic acid; 0.30 to 0.50 g/L potassium hydroxide; 0.05 to 0.10 g/L calcium chloride; 1.00 to 1.50 g/L magnesium sulfate; 3.00 to 4.00 g/L sodium phosphate monobasic; and 19.00 to 21.00 g/L pentastarch. For example, capping layer solution can have a density of 1.035 to 1.036 g/cm³ and can include 17.84 g/L D (+) raffinose, 4.67 g/L L-Histidine, 4.6 g/L sodium hydroxide, 35.83 g/L lactobionic acid, 0.393 g/L potassium hydroxide, 0.07 g/L calcium chloride dihydrate, 1.23 g/L magnesium sulfate heptahydrate, 3.45 g/L sodium phosphate monobasic, and 2% penta starch. Capping layer solution can be made by combining H-Phase II solution (80% by volume) with 10% penta starch (i.e., 100 g/L) (20% by volume).

### Purifying Islets Using Continuous Density Gradient Separation

Islets can be purified using continuous density gradient separation. Gradients can be prepared using iodixanol (OptiPrep™, Nycomed, Roskilde, Denmark) (density 1.32 g/cm³) and capping layer solution, cold storage solution, and/or high-density (HD) stock solution. HD stock solution can include 16.00 to 20.00 g/L raffinose; 4.00 to 6.00 g/L histidine; 4.00 to 5.00 g/L sodium hydroxide; 30.00 to 40.00 g/L lactobionic acid; 0.30 to 0.50 g/L potassium hydroxide; 0.05 to 0.10 g/L calcium chloride; 1.00 to 1.50 g/L magnesium sulfate; 3.00 to 4.00 g/L sodium phosphate monobasic; 15.00 to 25.00 g/L pentastarch; and 200 to 300 ml/L iodixanol. The density of the HD stock solution typically is 1.112±0.003 g/cm³. For example, HD stock solution can include 17.84 g/L D (+) raffinose, 4.67 g/L L-Histidine, 4.6 g/L sodium hydroxide, 35.83 g/L lactobionic acid, 0.39 g/L potassium hydroxide, 0.07 g/L calcium chloride dihydrate, 1.23 g/L magnesium sulfate heptahydrate, 3.45 g/L sodium phosphate monobasic, 20 g/L penta starch, and 250 mL/L iodixanol (Optiprep™). In some embodiments, HD stock solution also can include 8.00 to 12.00 U/mL of heparin and/or 8.00 to 12.00 μg/mL insulin.

A bottom density gradient solution having a density that ranges from 1.08 to 1.13 g/cm³ can be prepared by mixing HD stock solution and cold storage solution. A light density gradient solution having a density of 1.050 to 1.080 g/cm³ can be made by mixing iodixanol and cold storage solution, while a heavy density gradient solution having a density of 1.06 to 1.13 g/cm³ can be made by mixing cold storage solution and HD stock solution.

A continuous gradient can be made, for example, in a dual chamber gradient maker, by combining the light and heavy density gradient solutions. The bottom density gradient can be transferred to a cell processing bag for a cell separator such as the Cobe 2991 cell separator (Lakewood, Colo.), and the continuous gradient can be overlaid on the bottom density gradient. The resuspended tissue (as described above) can be placed on the continuous gradient followed by a capping layer solution then the gradient can be spun to separate the islets. Fractions can be collected and assayed for the presence of islets as described below. Fractions with islet purities (percentage of DTZ positive cells) >10% can be combined for culture.

### Culturing Purified Islets

Purified islets can be cultured using a chemically defined culture medium that is effective for maintaining viability of human pancreatic islets under culture conditions. Typically, islets are cultured at a temperature of 22° C. or 37° C. and an atmosphere of 95% air and 5% CO₂. In some embodiments, islets can be cultured in an atmosphere of room air. Viability of islets can be assessed using trypan blue or a fluorescent dye inclusion/exclusion assay. See, for example, Barnett et al., Cell Transplant. 2004; 13(5):481-8.

The chemically defined culture medium can include one or more of the following: insulin, zinc sulfate, selenium, transferrin, sodium pyruvate, HEPES (N-[2-Hydroxyethyl]piperazine-N′[2-ethanesulfonic acid]), HSA, and heparin. For example, the chemically defined culture medium can include 5.50 to 7.50 μg/mL insulin, 15 to 18 μM zinc sulfate, 5.50 to 7.50 ng/mL selenium (e.g., selenous acid), and 5.50 to 7.50 μg/mL transferrin (e.g., human transferrin). Such a culture medium further can include one or more of the following: 3 to 7 mM sodium pyruvate, 20 to 30 mM HEPES, 0.50 to 1.50 mg/mL HSA, 8.00 to 12.00 U/mL of heparin, 1 to 3 mM L-Alynyl-L-glutamine, and 4.50 to 6.50 μg/mL linoleic acid. Typically, when the cells are to be cultured under 95% room air and 5% CO₂, the chemically defined culture medium includes bicarbonate (e.g., 1.75 to 2.75 g/L such as 2.2 g/L). The bicarbonate concentration can be reduced if the cells are cultured in 100% room air. In some embodiments, the chemically defined culture medium also includes an antibiotic such as ciprofloxacin (Bayer Corporation).

In one embodiment, a chemically defined culture medium can be CMRL 1066 (Mediatech, Inc., Herndon, Va.) supplemented with 25 mM HEPES, 2 mM L-Alynyl-L-Glutamine, 5 mM sodium pyruvate, 1% (vol/vol), ITS additive (6.25 μg/mL human recombinant insulin, 6.25 μg/mL human transferrin, 6.25 ng/mL selenous acid, 1.25 mg/mL HSA, 5.35 μg/mL linoleic acid), 16.7 μM zinc sulfate, 20 μg/mL ciprofloxacin (Bayer Corporation) and 0.5% final concentration of 25% HSA. Human Insulin-like Growth Factor-I (IGF-I, GRO PEP Pty Ltd, Adelaide, South Australia) can be added to the islet culture. For example, 90 to 110 ng/mL (e.g., 100 ng/mL) of IGF-1 can be added to the culture.

Typically, the islets are cultured overnight at 37° C. then for an additional 1 to 3 days at 22° C. Pretransplant culture of islets can provide beneficial metabolic and immunologic effects. For example, culturing islets for two days can improve the metabolic efficacy of the cultured islets relative to freshly isolated islets. Pretransplant islet culture also can allow time for T-cell-directed immunosuppression to be achieved in the recipient before the transplant. Without being bound to a particular mechanism, achieving T-cell-directed immunosuppression may reduce islet-directed immune responses mediated by autoreactive, primed T cells to which the transplanted islets are immediately exposed. As described herein, delaying transplantation until two days after the initiation of therapy with T-cell-depleting antibodies prevents exposure of transplanted islets to the cytokine release associated, to varying degrees, with the first and second antibody infusions. Furthermore, pretransplant culture of islets allows quality control studies to be performed before the infusion of tissue.

### Cryopreservation of Islets

Purified islet cells can be cryopreserved by suspending the cells in a cryopreservative such as dimethylsulfoxide (DMSO) or ethylene glycol, or a mixture of cryopreservatives. See, for example, Miyamoto et al., Cell Transplant 2001; 10(4-5):363-71; Evans et al., Transplantation 1990; 50(2):202-206; and Lakey et al., Cell Transplant 1996; 5(3):395-404. Islet cells can be cryopreserved after purification or culture. Typically, the cryopreservative is added in a stepwise fashion and the islets are slow cooled to −40° C. then stored at −196° C. Islets can be rapidly thawed (e.g., in a 37° C. water bath) and assayed before use. Cryopreservation can allow for long-term storage of these cells for later transplantation or other purpose. Cryopreserving collections of purified populations of islets cells is particularly useful for producing an islet bank.

### Characterizing Preparations of Purified Islet Cells

Preparations of isogenic islet cells purified using the methods described herein typically result in successful transplants in at least 55% (e.g., at least 60%, 65%, 70%, 75%, 80%, 85%, 90%, or 95%) of the patients. A transplant is considered a success when a patient sustains insulin independence, normoglycemia, and freedom from hypoglycemia for at least one year after a single-donor islet transplant.

Preparations of purified islet cells can be assayed to confirm that the islets have sufficient potency to be transplanted. As used herein, “transplant potency” refers to an estimate of the probability that the preparation of islets can be successfully transplanted in a patient and is based on one or more of the following parameters: safety of the islet preparation, islet cell number, cellular composition of islet preparation, number of beta cells, insulin content, tissue volume, viability, ATP content, percent of islet equivalents recovered after cell culture, percent necrotic and apoptotic cells, glucose-stimulated insulin release, and oxygen consumption rate (OCR). For example, transplant potency can be estimated based on the ATP/DNA ratio, OCR/DNA ratio, and beta cell number. Preparations of purified islets that have at least a 60% probability of constituting a successful transplant are particularly useful.

Safety of an islet preparation can be determined by assaying for the presence of aerobic and anaerobic organisms and fungi, mycoplasma, and other adventitious agents (e.g., viruses) using known techniques. For example, a sample can be Gram stained to detect bacteria. Islet cells suitable for transplantation do not contain detectable organisms and are functionally sterile. Assessing safety also can include measuring endotoxin present in the preparation. Islet cell preparations suitable for transplant have an endotoxin content of 1.7 EU/mL (5 EU/kg recipient body weight) or less.

Islet cell number can be assessed by staining with DTZ and quantifying the size distribution of the stained cells using a light microscope with ocular micrometer. See, Ricordi et al., Acta Diabetol. Lat. 1990; 27:185-195. Islet volume can be calculated, based on the assumption that islets are spherical, and the number of islets is expressed in terms of islet equivalents (IE), with one IE equal to a 150 μm diameter islet. Preparations of islets containing at least 2.2×10⁵ IE (e.g., 2.7×10⁵, 3.5×10⁵, 4.5×10⁵, 5.5×10⁵, 7.0×10⁵, 9.0×10⁵, 1.1×10⁶, or 1.4×10⁶ IE) are particularly useful as 5,000 to 20,000 IE can be transplanted/kg recipient body weight. One IE can include from about 600 to about 8600 cells.

The cellular composition of islet preparations can be assessed using standard immunoassay methods. Antibodies that have binding affinity for insulin, glucagon, somatostatin, pancreatic polypeptide, amylase, and cytokeratin 19 can be used to identify β-, α-, δ-, pp-, acinar, and ductal cells, respectively. Such antibodies are commercially available, e.g., from DAKO, Carpinteria, Calif. or Sigma Chemical Co., St. Louis, Mo. Binding can be detected by labeling, either directly or indirectly, the antibody having binding affinity for the particular protein (e.g., insulin) or a secondary antibody that binds to such an antibody. Suitable labels include, without limitation, radionuclides (e.g., ¹²⁵I, ¹³¹I, ³⁵S, ³H, ³²P, ³³P, or ¹⁴C), fluorescent moieties (e.g., fluorescein, FITC, PerCP, rhodamine, or PE), luminescent moieties (e.g., Qdot™ nanoparticles supplied by the Quantum Dot Corporation, Palo Alto, Calif.), compounds that absorb light of a defined wavelength, or enzymes (e.g., alkaline phosphatase or horseradish peroxidase). Antibodies can be indirectly labeled by conjugation with biotin then detected with avidin or streptavidin labeled with a molecule described above. Methods of detecting or quantifying a label depend on the nature of the label and are known in the art. Examples of detectors include, without limitation, x-ray film, radioactivity counters, scintillation counters, spectrophotometers, colorimeters, fluorometers, luminometers, and densitometers. Immunological assays can be performed in a variety of known formats, including sandwich assays, competition assays (competitive RIA), or bridge immunoassays. See, for example, U.S. Pat. Nos. 5,296,347; 4,233,402; 4,098,876; and 4,034,074.

The number of beta cells can be calculated based on the total DNA content and proportion of beta cells identified in the cellular composition sample. One IE can include from about 145 to 4000 beta cells. Preparations of islet cells that contain at least 1×10⁶ beta cells/kg body weight of recipient (i.e., 4.5×10⁷ beta cells for a 45 kg recipient, 5×10⁷ beta cells for a 50 kg recipient, and 5.5×10⁷ beta cells for a 55 kg recipient) can be used. Preparations containing higher numbers of beta cells (e.g., at least 2×10⁶ beta cells/kg body weight of recipient, at least 3.5×10⁶ beta cells/kg body weight of recipient, or at least 5.0×10⁶ beta cells/kg body weight of recipient) are particularly useful. For example, as shown in Example 3, preparations containing at least 3.5×10⁶ beta cells/kg body weight of recipient (i.e., about 1.58×10⁸ beta cells for a 45 kg recipient, about 1.75×10⁸ beta cells for a 50 kg recipient, and about 1.9×10⁸ beta cells for a 55 kg recipient) can sustain insulin independence for at least one year.

Insulin content can be assessed using an immunoassay, e.g., the Human Insulin Enzyme Immunoassay (EIA) kit from Mercodia, Sweden, and corrected for the DNA content. Pico Green can be used to assess DNA content. In the Pico Green method, islet cells can be lysed with a solution containing ammonium hydroxide and a non-ionic detergent. Pico Green can be added to the sample and incubated in the dark. Samples are read on a fluorometer with an excitation of 480 nm and an emission of 520 nm and compared with a standard curve. Typically, one IE can include from about 4 to about 60 ng of DNA.

Tissue volume of the preparation refers to the volume of the islet cell pellet before transplant. Islet cells can be collected in a pre-weighed tissue culture flask and the islets can be allowed to sediment to a bottom corner of the flask over a period of time (e.g., 5 minutes). The medium can be removed from the flask and the mass recorded. Suitable preparations of islet cells have a volume of 10 mL or less (e.g., 8 mL or less, 7.0 mL or less, 5 mL or less, 3 mL or less, or 2 mL or less).

ATP content of islet cell preparations can be assessed via high performance liquid chromatography (HPLC) or by using an immunoassay (e.g., an ATP Determination Kit from Invitrogen Corp., Carlsbad, Calif.). In either method, samples can be prepared using the methods of Micheli et al. Clin. Chem. Acta 1993, 220:1-17 in which trichloroacetic acid is used to extract the ATP and a freon/amine solution is used to neutralize the sample. Preparations of islet cells that have at least 76 pmol ATP/μg DNA (e.g., at least 80, 90, 100, 110, 150, 175, 190, or 193), as measured by HPLC, are particularly useful for transplants.

A fluorescent dye inclusion/exclusion assay can be used to assess viability. See, for example, London et al., Hormone & Metabolic Research—Supplement 1990; 25:82-87. For example, fluorescein diacetate and propidium iodide (PI) can be used to assess viability. Fluorescein diacetate is dissociated by intracellular enzymes into free fluorescein, which fluoresces green under blue light excitation (490 nm) and provides evidence that the cells are alive and metabolically active. If the cell membrane has been damaged, PI can enter into the cell, intercalate into the nuclear DNA, and fluoresce red under green light excitation (545 nm). The proportion of green (viable) and red (dead) cells gives an indication of viability of the islet preparation. Alternatively, SYTO-13/ethidium bromide (SYTO/EB) and calcein AM/ethidium homodimer (C/EthD) fluorescent staining can be used to assess viability. See, for example, Barnett et al., Cell Transplant. 2004; 13(5):481-8. Preparations of islets that contain at least 70% (e.g., at least 75%, 80%, 85%, 90%, 95%, or 97%) viable cells are particularly useful for transplants.

The percent of IE recovered after culture can be determined using DTZ as described above. Preparations of islets in which at least 70% (e.g., least 75%, 80%, 85%, 90%, or 95%) of the IE were recovered after culture are particularly useful for transplants.

The percent necrotic and apoptotic cells can be assessed using known methods. For example, apoptosis can be assessed by examining DNA fragmentation. For example, a Cell Death Detection ELISA^{Plus} (Roche Biochemicals, Indianapolis, Ind.) can be used to detect cytoplasmic histone-associated DNA fragments. Preparations of islets in which 30% or less (e.g., 25%, 20%, 15%, 10%, 5%, or less) of the cells are apoptotic or necrotic are useful for transplants.

Glucose-stimulated insulin release is a measure of the functional capacity of the preparation. Standard techniques for static incubation and assessment of insulin release corrected for DNA content are utilized to determine the functional capacity of the islets. Ricordi et al., Acta Diabetol. Lat. 1990; 27:185-195. A stimulation index is calculated by dividing insulin release at 16.7 mM glucose by insulin release at 1.7 mM glucose. Preparations of islets that have a stimulation index of >1 (e.g., >4, >7, >10, >14, >17, or >27) are particularly useful for transplants.

OCR can be measured using an OCR chamber (e.g., from Instech Laboratories, Inc., Plymouth Meeting, Pa.). See, for example, Papas et al., Cell Transplant. 2003; 12: 177; Papas et al., Cell Transplant. 2003; 12: 176; and Papas et al., Cell Transplant. 2001; 10: 519. Preparations of islets having an OCR of greater than >75 nmol/min/mg DNA (e.g., greater than >100, >150, >200, or >230 nmol/min/mg DNA) are particularly useful for transplants.

### Transplantation

Islet cells can be transplanted into, for example, the portal vein of a patient using surgical techniques such as minilaparotomy or percutaneous transhepatic portal venous catheterization. Prior to transplant, patients can undergo induction immunosuppression using different therapy regimens. Patients also can undergo post-transplant immunosuppression regimens. For example, induction therapy can include treatment with rabbit antithymocyte globulin (RATG), daclizumab, and etanercept (i.e., soluble tumor necrosis factor (TNF) receptor). RATG is a potent induction agent and also interferes with leukocyte responses to chemotactic signals and inhibits the expression of integrins required for firm cellular adhesion. Selective inhibition of TNF-α in the peritransplant period may be able to promote reversal of diabetes after marginal-mass islet transplants. Post-transplant, the function of engrafted islets may be enhanced by replacing or minimizing tacrolimus at 1 month post-transplant. See Example 3 of the specification.

Another example of an induction therapy can include use of anti-CD3 mAb hOKT3γ1 (Ala-Ala), which can inactivate autoreactive, primed, islet-directed T cells immediately posttransplant. Anti-CD3 mAb, hOKT3γ1 (Ala-Ala), is a humanized antibody that retains the binding region of OKT3 but replaces the murine framework with human amino acids. In addition, the human IgG1 Fc is mutated to prevent binding to the Fc receptor (FcR). Clinically, this engineered antibody has proven effective in preserving residual beta-cell function in new-onset type 1 diabetes. In addition, the hOKT3γ1 (Ala-Ala) reversed kidney graft rejection. This dual activity against both autoreactive and alloreactive T cell responses occurred with markedly fewer side effects, as compared with the parental OKT3 antibody. See Example 4 of the specification.

### Kits and Articles of Manufacture

Compositions of the invention (e.g., capping layer solution, cold storage solution, H-Phase II solution, cold transport solution, Phase I solution, chemically-defined culture medium, or HD stock solution) can be combined with packaging material and sold as an article of manufacture or a kit. For example, an article of manufacture or kit can include the solutions used to isolate islets by the automated method for pancreatic tissue dissociation or can include the solutions used to purify islets by continuous density gradient separation. An article of manufacture or kit also can include a chemically defined culture medium for culturing purified islets. In some embodiments, an article of manufacture can include solutions for isolating, purifying, and culturing islets. Typically, the packaging material included in a kit includes instructions or a label describing how the compositions can be used (e.g., to isolate, purify, or culture islets). Components and methods for producing such kits are well known.

In some embodiments, purified populations of islet cells (e.g., human isogenic islets that are substantially free of pancreatic islet cells non-isogenic to the human islets) can be included in an article of manufacture or kit. In still other embodiments, the article of manufacture or kit can include one or more cryopreservatives or pharmaceutically acceptable carriers. For example, an article of manufacture or kit can include DMSO.

Cryopreserved preparations of islet cells also can be combined in a kit with packaging material. The packaging material can include instructions or a label describing how the purified populations of islet cells can be used, e.g., for transplant into a patient or in an islet bank. In some embodiments, collections of purified populations of islet cells can be combined in an article of manufacture or kit. For example, a collection of at least five (e.g., at least 10, 15, 20, 40, or 50) cryopreserved preparations of islet cells can be combined as an article of manufacture or kit.

The invention will be further described in the following examples, which do not limit the scope of the invention described in the claims.

### EXAMPLES

The description of the development, characterization, and release testing of human allogeneic pancreatic islet tissue follows 21 CFR and “Guidance for Human Somatic Cell Therapy and Gene Therapy,” released by the U.S. Department of Health and Human Services, Food and Drug Administration, Center for Biologics Evaluation and Research in March, 1998.

### Example 1

### Isolation and Purification of Islets

In general, preparations of allogeneic islets of Langerhans for transplantation were obtained from cadaveric human donor pancreata using the following steps: 1) Procuring and preserving donor pancreata; 2) Isolating islets using the automated method for pancreatic tissue dissociation; 3) Purifying islets using continuous density gradient separation; and 4) Culturing islets. Each of these steps is explained in detail below.

1. Procuring and Preserving Donor Pancreata

A. Donor Selection Criteria: Pancreata were obtained from male and female, brain-dead, heart-beating multi-organ donors who were less than 50 years of age. Upon receipt, donor records were screened to ensure compliance with federal regulation 21 CFR 1270, “Human Tissue Intended for Transplantation” for use as a tissue donor. Required tests include HIV-1, HIV-2, HTLV-1, HTLV-2, HBsAg, and HCV. The volume and type of any blood or colloid products infused within 48 hours and or crystalloid products infused within one hour of the donor's death or donor blood sample time also were required to determine plasma dilution. Donor medical histories included information about the behavioral and high risk criteria; tissue was not accepted for transplantation from donors who have any exclusionary risk factors outlined in 21 CFR 1270 and the Guidance for Industry document titled “Screening and Testing of Donors of Human Tissue Intended for Transplantation.” Additional exclusion criteria included history of diabetes, cold storage time of the donor pancreas exceeding 8 hours, systemic infection, extracranial tumor, and risk factors for AIDS. Donors also were screened for Cytomegalovirus (CMV-IgG) titers.

B. Tissue Typing: Minimum matching criteria included negative serum cross match for T cells and ABO compatibility. HLA-A and HLA-B typing was performed using standard serological techniques. HLA-DR and DQ typing of donor and recipient pairs was performed by molecular methodology using polymerase chain reaction (PCR) with sequence-specific oligonucleotide probes. The degree of HLA antigen matching was not considered a prerequisite for the allocation of islets to one particular patient.

C. Donor Pancreas Procurement and Preservation:

Pancreas procurement from multi-organ donors was accomplished utilizing standard techniques previously developed for pancreas procurement prior to whole organ pancreas transplantation. Donor pancreata were received that were recovered and preserved using the solutions shown in Table 1 before the initiation of the islet processing.

TABLE 1
Solutions for Preserving Pancreata

| Preservation Step | Solution |
|---|---|
| Arterial Perfusion | University of Wisconsin (UW) Solution (ViaSpan ®, |
| | DuPont Pharma, Wilmington, Delaware) |
| Pre-Cold Storage | None, UW Solution, or Modified-UW Solution |
| Ductal Injection | (PICS or Pancreas/Islet Cold Storage solution, |
| | Protide Pharmaceuticals, St. Paul, MN) |
| Cold Storage | UW Solution or Perfluorodecalin (FluoroMed, L.P., |
| Preservation | Round Rock, TX)/UW Solution (Two-Layer) or |
| | Perfluorodecalin/Modified-UW Solution (Two- |
| | Layer) |

Donor pancreata were preserved by the two-layer method in a custom-made sterile organ shipping container. Cold ViaSpan® or modified-UW solution (500 mL) was poured on top of 500 mL of cold perfluorodecalin (the specific gravity of perfluorodecalin is greater than ViaSpan® or modified-UW solution) and an interface formed between the two solutions to form two layers. The modified UW solution contains 0.39 g/L potassium hydroxide, 3.45 g/L monosodium phosphate-monohydrate, 0.074 g/L calcium chloride-dihydrate, 1.233 g/L magnesium sulfate-heptahydrate, 35.83 g/L lactobionic acid, 4.656 g/L L-histidine, 17.84 g/L raffinose, 4.600 g/L sodium hydroxide, 20.00 g/L penta starch, 1.34 g/L adenosine, and 0.92 g/L glutathione. The lid was closed and the perfluorodecalin was oxygenated for 40-60 minutes by filtering medical grade oxygen through a 0.2 μm filter (Gelman Sciences, Ann Arbor, Mich.) and the inlet port at a rate of 2.5 L/min. The lid was temporarily removed and the pancreas was placed in the container and kept near the interface via a stainless steel mesh plate attached to the lid of the container.

2. Islet Isolation Using the Automated Method for Pancreatic Tissue Dissociation (Ricordi et al., Diabetes 1988; 37:413-420):

Upon arrival, each pancreas was inspected for package integrity and then removed from the container and rinsed with 500 mL of cold transport solution. The cold transport solution contained 1.122 g/L potassium hydroxide, 2.722 g/L potassium phosphate monobasic, 0.037 g/L calcium chloride dihydrate, 1.233 g/L magnesium sulfate heptahydrate, 9.11 g/L D-mannitol, 19.629 g/L D-gluconic acid sodium salt, 4.656 g/L L-histidine, 0.611 g/L nicotinamide, and 0.55 g/L sodium pyruvate. The pancreas was placed in a sterile tray with cold transport solution, some of the extraneous fat, and non-pancreatic tissue was carefully dissected and discarded. Some of the excess fat was retained to minimize leaking during the distension. The pancreas then was placed in a topical antibiotic solution of 80 mg gentamicin (Elkins-Sinn, Inc.), 1 g Cefazolin (SmithKline Beecham Pharmaceutical), and 100 mg amphotericin-B (Apothecon®) in a volume of 150 mL of cold transport solution. After a 5-minute incubation period, the pancreas was serially rinsed in two beakers containing 500 mL of phenol red-free Hanks' Balanced Salt Solution (8.00 g/L NaCl, 0.05 g/L Na₂HPO₄H₂O, 0.4 g/L KCl, 0.06 g/L KH₂PO₄, 0.98 g/L MgSO₄, 0.14 g/L CaCl₂, and 1.00 g/L D-glucose, Mediatech, Inc.). The pancreas was weighed and placed in a sterile tray containing 500 mL of cold transport solution and frozen saline.

After dividing the pancreas at the neck, the transport solution was poured off and the tray containing the pancreas was removed from the ice for the distension (enzyme loading) procedure. The following steps were performed on both the ‘body and tail’ and ‘head’ of the pancreas. The pancreatic duct was cannulated with an angiocatheter (16-20 gauge) and the pancreas was perfused under controlled conditions while maintaining constant pressure of 80 mmHg (27 mL/min) for the first 4 minutes and then increasing the pressure to 180 mmHg (60 mL/min) for the remainder of the distension procedure. Lakey et al., Cell Transplant. 1999; 8:285-292. The perfusion solution contained 1-2 mg/mL (depending on enzyme activities of a given lot) Liberase™ HI (Roche Molecular Biochemicals, Indianapolis, Ind.), 0.1 mg/mL Pefabloc® SC PLUS (Roche Molecular Biochemicals, Indianapolis, Ind.), and 10 U/mL heparin dissolved in Phase I solution (5.466 g/L D-mannitol, 0.600 g/L NaOH, 6.136 g/L NaCl, 0.325 g/L KOH, 0.110 g/L CaCl₂ 2H₂O, 0.197 g/L MgSO₄ 7H₂O, 3.45 g/L Na H₂PO₄H₂O). Liberase™ HI is reconstituted >20 minutes, but <2 hours, before use by adding 15 mL sterile water to the powder and mixing without creating air bubbles. The reconstituted Liberase™ HI then was added to the Phase I solution. Prior to perfusion, the collagenase solution was filtered through a 0.2 micron cellulose acetate bottle top filter. After 10-20 minutes of cold perfusion, the pancreas was further trimmed of remaining capsule and placed into the dissociation chamber.

Both the ‘body and tail’ and ‘head’ portion of the distended pancreas were cut in half and all parts placed in a sterile stainless steel chamber (Wahoff et al., Ann. Surg. 1995; 222:562-579, also known as a Ricordi chamber). Collagenase that “leaked” from the distended pancreas was added, and the remaining system volume filled with Phase I solution. The circulation system for digestion is shown in FIG. 1A and contains, in addition to the Ricordi chamber, a stainless steel coil for heat exchange, six (6) tubes with small diameter (Master Flex tubing, size 16), four (4) tubes with large diameter (Master Flex tubing, size 17), steel 3-way stopcock for sampling, four (4) plastic clamps, 250 mL conical tube, tri-pour graduated disposable beaker, 1000 mL, bell-shaped plastic cover, two (2) T-connectors, (1) T-connector with luer lock port, and one (1) Y-connector, 18 inch steel ring stand with two arms, Ismatec pump, Mon-a-therm temperature monitor and sensor, and water bath.

Initially, 300 mL of Phase I solution were added to the 1 L beaker of the digestion circuit and the flow rate was set to 225 mL/min. The system was filled with Phase I solution and air was evacuated from the system. FIG. 1B shows the flow direction from the 1 L beaker to the chamber and from the chamber to the 250 mL conical tube. After the 250 mL conical tube reached the 75 mL mark, the pump was stopped and the clamps were adjusted to recirculate. The flow rate was set to 100 mL/min and Phase I (recirculation phase) was started. FIG. 1C shows the flow direction from the 250 mL conical tube to the chamber and from the chamber to the 250 mL conical tube. The collagenase solution was recirculated during Phase I at 25° C. to 34° C. (peak temperature not exceeding 32° C.-34° C.) as the chamber was agitated. The chamber was gently rocked for five minutes then the chamber was shaken for maximum agitation. Samples (2 mL) were taken at regular intervals to monitor, via inverted microscope, the breakdown of the pancreas. The initial sample was taken when the tissue started appearing in the stream, typically after about 8 minutes. The “switch” from Phase Ito Phase II (collection phase) occurs when there is an increase in the amount of tissue liberated from the chamber, most or all of the islets are free of the surrounding acinar tissue, intact islets are observed, and the acinar tissue becomes finer (small cell clusters).

Once the switch point was reached, the recirculation beaker and the heating circuit were bypassed (see FIG. 1D) and the islet isolation was continued in a system in which the temperature was progressively decreased to 10-30° C. to slow the digestion and the collagenase was diluted with Phase II solution (RPMI 1640, catalog #99-595-CM, Mediatech, Inc., Herndon, Va.) that was pumped through the system at 200 mL/min. See FIG. 1E. The digest containing the free islets was collected first in four 1 L Erlenmeyer flasks followed by 250 mL conical tubes. The first Erlenmeyer flask was pre-filled with 675 mL of Phase II solution and 75 mL of 25% human serum albumin (HSA). The second and third flasks were pre-filled with 450 mL Phase II solution and 50 mL of 25% HSA. The fourth flask was pre-filled with 225 mL Phase II solution and 25 mL of 25% HSA. The remaining 250-mL conical tubes were pre-filled with 15 mL of 25% HSA. When collection reached conical tube #20, shaking of the chamber was stopped and the chamber was inverted and emptied. At regular intervals during Phase II of the digest, samples were taken and stained with diphenylthiocarbazone (DTZ) (see Example 2); the percentage of free islets, the degree of fragmentation, and the condition of the acinar tissue were noted. The conical tubes were centrifuged at 800 rpm (120×g) and 8° C. for 3 minutes with full brake (1^{st} washing step). The pellets were subsequently resuspended in cold storage solution (17.835 g/L D (+) raffinose, 4.656 g/L L-histidine, 4.600 g/L NaOH, 35.830 g/L lactobionic acid, 0.393 g/L KOH, 0.393 g/L CaCl₂ 2H₂O, 1.233 g/L MgSO₄ 7H₂O, 3.450 g/L NaH₂PO₄H₂O, 2% penta starch, 10 U/mL heparin, and 10 μg/mL insulin), combined into a 1 L Erlenmeyer flask, and kept cold.

Once the entire digest was collected, the combined pellets were distributed into four 250-mL conical tubes. The tubes were centrifuged at 1000 rpm (220×g) and 8° C. for 3 minutes (2^{nd} washing step) and the supernatant was evacuated. If the pellets were not tightly packed due, for example, to extracellular debris and/or DNA, the pellets were washed until packed tightly (1 to 3 more times). The pellets were transferred to a 400 mL sterile beaker tared on a scale. The pellets were resuspended by adding cold storage solution to 200 g and stirring with a sterile 10 mL disposable glass pipette along the wall of the beaker. The resuspended digest was poured from the first 400 mL beaker to a second 400 mL beaker. Care was taken to resuspend the tissue evenly and ensure that there was no clumping of the tissue. If the tissue clumped together, the tissue was washed again in cold storage solution. Two 100 μL samples were taken for determination of islet cell number. In preparation for the purification step, pellets ≦20 mL were kept in one tube. Pellets ≧20 mL were placed in separate tubes so there was no more than 20 mL loaded per cobe run.

3. Islet Purification Using Continuous Density Gradient Separation:

Prior to islet purification, test gradients were performed on a small volume of tissue to determine the density distribution of acinar and islet tissue. These findings were used to determine the densities of the continuous density gradient to be used on the Cobe 2991 (Gambro BCT, Lakewood, Colo.) for islet purification. Gradient solutions having densities ranging from 1.065 to 1.110 were prepared by mixing cold storage solution (density 1.035) and iodixanol (OptiPrep™, Mediatech, Inc., Herndon, Va.) (density 1.320) or high-density (HD) stock solution (density 1.112) as shown in Table 2. HD stock solution contains 17.835 g/L D (+) raffinose, 4.656 g/L L-Histidine, 4.6 g/L NaOH, 35.83 g/L lactobionic acid, 0.393 g/L KOH, 0.074 g/L CaCl₂ 2H₂O, 1.233 g/L MgSO₄ 7H₂O, 3.45 g/L NaH₂PO₄H₂O, 20 g/L penta starch, and 250 mL/L iodixanol (Optiprep™)

TABLE 2
Preparation of Gradient Solutions

| | | Stock solutions | | Check | |
|---|---|---|---|---|---|
| Vol | Desired | Cold storage (g) | Optiprep (g) | Vol. | |
| (mL) | density | 1.035 | 1.320 | (mL) | (g) |
| 40.0 | 1.065 | 37.042 | 5.558 | 40.0 | 42.600 |
| 40.0 | 1.070 | 36.316 | 6.484 | 40.0 | 42.800 |
| 40.0 | 1.075 | 35.589 | 7.411 | 40.0 | 43.000 |
| | | | HD-stock (g) | | |
| 40.0 | 1.080 | 17.205 | 25.995 | 40.0 | 43.200 |
| 40.0 | 1.085 | 14.517 | 28.883 | 40.0 | 43.400 |
| 40.0 | 1.090 | 11.829 | 31.771 | 40.0 | 43.600 |
| 40.0 | 1.095 | 9.140 | 34.660 | 40.0 | 43.800 |
| 40.0 | 1.100 | 6.452 | 37.548 | 40.0 | 44.000 |
| 40.0 | 1.105 | 3.764 | 40.436 | 40.0 | 44.200 |
| 40.0 | 1.110 | 1.075 | 43.325 | 40.0 | 44.400 |

Five mL of each gradient solution were placed in a pre-labeled, 15 mL conical tube then 1 mL of capping layer solution (17.835 g/L D (+) raffinose, 4.656 g/L L-Histidine, 4.6 g/L NaOH, 35.83 g/L lactobionic acid, 0.393 g/L KOH, 0.074 g/L CaCl₂ 2H₂O, 1.233 g/L MgSO₄ 7H₂O, 3.45 g/L NaH₂PO₄H₂O, and 2% penta starch, density 1.035 to 1.036) was placed on top of each gradient solution. Digested material (12 mL) obtained just after the switch point from phase I to phase II was spun at 800 rpm for 1 minute in Beckman centrifuge and the supernatant discarded. After resuspending the pellet in 1.2 mL of cold storage solution, 200 μL of the resuspended sample were loaded onto the top of each tube. The tubes were spun in a Beckman centrifuge for 3 minutes at 1500 rpm (400×g). The location of the pelleted material was recorded and used to determine the densities of the continuous density gradient to be used on the Cobe 2991 for islet purification. The hydraulic system of the Cobe 2991 apparatus was primed and checked according to the manufacturer's specifications before continuing with the density gradients.

Continuous density gradients were performed as follows. In the hood in the clean cold room, the centrifuged pellet from step 2) above was brought to a total of 120 mL with 20 mL of 25% HSA and capping layer solution. The bottom density gradient (120 mL, range 1.080 to 1.130 g/cm³) was made by mixing HD stock solution (H) and Cold storage solution (C) as indicated by the following equations:

$$weight ( g ) of ( H ) = density ( g / mL ) of ( H ) \times desired volume ( mL ) \times \frac{desired density ( g / mL ) - density ( g / mL ) of ( C )}{density ( g / mL ) of ( H ) - density ( g / mL ) of ( C )}$$

$$weight ( g ) of ( C ) = density ( g / mL ) of ( C ) \times desired volume ( mL ) \times \frac{density ( g / mL ) of ( H ) - desired density ( g / mL )}{density ( g / mL ) of ( H ) - density ( g / mL ) of ( C )}$$

The bottom density gradient was drained into a 600 mL transfer pack (Baxter Health Care) and then transferred to the Cobe 2991 cell processing bag (Cobe, Lakewood, Colo.) previously placed into the Cobe 2991 cell separator. The Cobe 2991 was started, the speed increased to 1800 rpm, and the bottom density gradient overlayed with a continuous density gradient. The continuous gradient was made by a dual chamber gradient maker using a light density gradient (125 mL, cold storage solution plus iodixanol, range 1.050 to 1.080 g/cm³) and a heavy density gradient (125 mL, cold storage solution plus HD stock, range 1.060 to 1.125 g/cm³). The light density gradient was made by mixing Optiprep (O) and Cold storage solution (C) as indicated by the following equations:

$$weight ( g ) of ( O ) = density ( g \text{/} mL ) of ( O ) \times desired volume ( mL ) \times \frac{desired density ( g \text{/} mL ) - density ( g \text{/} mL ) of ( C )}{density ( g \text{/} mL ) of ( O ) - density ( g \text{/} mL ) of ( C )}$$

$$weight ( g ) of ( C ) = density ( g \text{/} mL ) of ( C ) \times desired volume ( mL ) \times \frac{density ( g \text{/} mL ) of ( O ) - desired density ( g \text{/} mL )}{density ( g \text{/} mL ) of ( O ) - density ( g \text{/} mL ) of ( C )}$$

The heavy density gradient was made by mixing HD stock solution (H) and Cold storage solution (C) based on the equations used for the bottom density gradient. The densities were adjusted for each islet isolation as suggested by test gradients.

The resuspended tissue was pumped slowly onto the continuous gradient (50 mL/min) followed by the capping layer solution (density 1.035 g/cm³). After spinning at 1800 rpm for 3 minutes with a COBE “super out” setting rate of 100 mL/min and volume of 600 mL, an initial 125 mL fraction was collected (waste) followed by twelve (12) 25 mL fractions, which were screened for the presence of islets. Fractions with islet purities (percentage of DTZ positive cells)>10% were combined for culture.

4. Cell Culture Procedures

The islet suspension obtained from the density gradients was cultured free-floating in an atmosphere of 95% air and 5% CO₂ in 175 cm² tissue culture flasks in CMRL 1066 (Mediatech, Inc., Herndon, Va.) supplemented with 25 mM HEPES, 2 mM L-Alynyl-L-Glutamine, 5 mM sodium pyruvate, 1% (vol/vol), ITS additive (6.25 μg/mL human recombinant insulin, 6.25 μg/mL human transferrin, 6.25 ng/mL selenous acid, 1.25 mg/mL human serum albumin, 5.35 μg/mL linoleic acid), 16.7 μM zinc sulfate, 20 μg/mL ciprofloxacin (Bayer Corporation) and 0.5% final concentration of 25% HSA all filtered with a 0.2 micrometer filter. After islets were placed in supplemented CMRL 1066, human Insulin-like Growth Factor-I (IGF-I, GRO PEP Pty Ltd, Adelaide, South Australia) was added at a final concentration of 100 ng/mL. See, Paraskevas et al., Transplantation 69(8), 5377. 4-27-2000. Islet preparations with a purity ≧70% were cultured overnight at 37° C. and for an additional 24 to 72 hours at 22° C. Islet preparations with purity <70% were diluted appropriately to give the same tissue concentration as pure preparations and were cultured at 22° C. until transplant.

Prior to transplantation, the islets were collected from the tissue culture flasks, washed three times in sterile filtered transplant medium [phenol-red free CMRL-1066 (Mediatech, Inc.) supplemented with 2.5% HSA and 25 mM HEPES (Sigma) to remove cellular debris, tissue culture media, and soluble proteolytic enzymes. The islets were suspended in 200 mL of transplant medium with the addition of heparin (APP Inc., Schaumburg, Ill.) at 70 U/kg recipient body weight, and collected into a 600-mL Fenwal Transfer Pack Container. The container was labeled as “Allogeneic Islets of Langerhans” and also included recipient information, reference number of the allogeneic islet preparation, and processing time and date of the allogeneic islet preparation.

### Example 2

### Identification and Characterization of Islets

The following methods can be used to identify and characterize islets.

Diphenylthiocarbazone (DTZ) staining: DTZ (100 mg) is dissolved in 10 mL dimethylsulfoxide (DMSO) and diluted with 40 mL Hanks Balanced Salt Solution (Mediatech, Herndon, Va.). Two representative aliquots, 100 μL each, are taken using a Drummond pipette from both the digest (before density gradient separation) and post-density gradient islet cell suspensions (100 mL) and incubated with DTZ. Using a light microscope with an ocular micrometer, the size distribution of the islets is quantified within a range of 50 to >400 μm by two independent observers (ranges: 50-100, 100-150, 150-200, 200-250, 250-300, 300-350, 350-400 and >400). See, Ricordi et al., Acta Diabetol. Lat. 1990; 27:185-195. Islet volume is calculated, based on the assumption that islets are spherical, and the number of islets is expressed in terms of islet equivalents (IE), with one IE equal to a 150 μm islet.

Insulin Content Insulin content is measured after extracting the islets in 2 mmol/L acetic acid containing 0.25% BSA. Samples are sonicated in acetic acid, centrifuged (800×g, 15 minutes), then supernatants are collected and stored at −20° C. until assayed for insulin content. The same samples also can be assayed for other pancreatic hormones.

DNA Content: To determine the DNA content, the pelleted cells are lysed with 500 μL of AT-Extraction solution (AT), which is prepared by mixing 33.33 mL of 1N ammonium hydroxide, 1 mL of Triton X-100, and 465.7 mL of ddH₂O. Each sample is sonicated for 10 seconds in ice water.

One hundred μL of AT solution are added to the first 8 wells of a 96-well microtiter plate and 100 μL of a dsDNA standard (2 μg/mL calf thymus DNA in TE buffer (10 mM Tris-HCl, 1 mM EDTA, pH to 7.5)) are placed in the first well of the 96-well plate (final concentration is 1 g/mL). Seven 1:2 dilutions are performed with 100 μL to produce standards of 1, 0.5, 0.25, 0.125, 0.0625, 0.0313, 0.0156 and 0 μg/mL.

After adding 100 μL of sample to the remaining wells, 100 μL of freshly prepared PicoGreen (0.5 μL PicoGreen and 99.5 μL TE Buffer per well) are added to each sample and standard. The plate is incubated for 15 minutes in the dark at room temperature and read on a fluorometer with an excitation of 480 nm and an emission of 520 nm.

Cellular composition of islet preparations: To quantitate the cellular composition of islet preparations for transplantation, islets are dissociated into single cells and fixed to microporous transparent membranes. The cells are stained immunocytochemically with antibodies that characterize endocrine and exocrine components. Primary antibodies include polyclonal guinea pig anti-swine insulin (DAKO, Carpinteria, Calif.), monoclonal mouse anti-glucagon, clone K79BB10 (Sigma, Saint Louis, Mich.), polyclonal rabbit anti-human somatostatin (DAKO), polyclonal rabbit anti-human pancreatic polypeptide (DAKO), rabbit anti-human α-amylase antiserum (Sigma), and monoclonal mouse anti-human cytokeratin 19, clone RCK108 (DAKO). Biotinylated secondary antibodies are purchased from Vector Laboratories (Burlingame, Calif.). The dissociated islet cells are stained for the presence of insulin, glucagon, somatostatin, pancreatic polypeptide, amylase, and cytokeratin 19 in order to identify β-, α-, δ-, pp-, acinar, and ductal cells, respectively. The immunocytochemical technique employs a primary antibody, followed by a biotinylated secondary antibody. Next, a preformed avidin and biotinylated horseradish peroxidase complex (termed ABC) is added, followed by chromagen 3-amino-9-ethyl carbazole (AEC) to localize peroxidase and produce a red color reaction in positive cells. Negative cells are visualized with Harris's hematoxylin counter stain nuclei that appear dark blue. The light microscopic view of each sample is photographed using a digital camera. The number of cells staining positive or negative is counted visually (ACD Photo Enhancer software) and the percentage of each type of cell for a specific primary antibody is calculated based on the ratio of positive to negative cells in the same area of the membrane.

When considering the DNA recovery per islet graft, the DNA content of human islet cells (6.0 pg/cell), and the percentage of insulin-positive cells, the following equation is used to calculate total beta cell mass in each islet graft:

$$\frac{Total DNA content}{6.0 pg DNA \text{/} cell} \times \frac{\% insulin positive cells}{100} = Number of beta cells recovered per graft$$

ATP Content: Two aliquots of 500 IE from islet culture are transferred to a 1.5 mL microcentrifuge tube. Cold phosphate-buffered saline (PBS) (1 mL) is immediately added and the tube is swirled. After a quick spin to pellet the cells, the supernatant is discarded and the wash with PBS is repeated. The tube is vortexed briefly to loosen the cells and 100 μL of cold 10% trichloroacetic acid (TCA) are added, followed by additional vortexing for 10 seconds and incubation on ice for 5 minutes. This step is repeated two more times. After the final incubation on ice, the sample is centrifuged at 14,000×g at 4° C. for 5 minutes. Approximately 100 μL of the supernatant are transferred to a clear 1.5 mL microcentrifuge tube. The pellet is stored at less than 4° C. for DNA analysis.

The supernatant is neutralized by adding 150 μL (1.5× volume) of cold Freon/Amine (0.5M tri-n-octylamine in freon) solution. The sample is vortexed for 10 seconds then centrifuged at 14,000×g at room temperature for 10 minutes. Approximately 70 μL of the top aqueous layer are transferred to a clear 1.5 mL microcentrifuge tube by placing the pipette tip vertically and horizontally in the center of the top aqueous layer without touching the bottom layer. The sample is stored at −80° C. for ATP analysis using an ELISA kit (ATP Determination Kit, Invitrogen Corp., Carlsbad, Calif.).

Oxygen Consumption Rate (OCR) Measurements: Islets are suspended in 250 μL of pre-warmed (37° C.) CMRL-1066 (Mediatech, Herndon, Va.) then transferred into the OCR chamber, which has been equilibrated to 37° C. The chamber (Instech Laboratories, Inc., Plymouth Meeting, Pa.) is sealed and excess fluid is expelled from a port. The solution pO₂ decreases with time. As long as pO₂ in the medium is far above the Michaelis constant for oxygen consumption, the data fits a straight line and OCR is calculated from the slope using the relation OCR=SαV_{ch}, where S is the slope, α is the Bunsen solubility coefficient, and V_{ch} is the chamber volume.

Potency: In order to determine the functional capacity of the preparation for transplantation, aliquots of islets cultured overnight at 37° C. are studied. On the subsequent morning, standard techniques for static incubation and assessment of insulin release corrected for DNA content are utilized to determine the functional capacity of the islets.

The following solutions are used to determine insulin release from islets. Solution A is made by mixing 500 mL RPMI-1640 (without glucose and sodium bicarbonate) with 2.98 g HEPES (25 mM final concentration) and 0.5 g BSA (0.1%), adjusting the pH to 7.2, and filtering using a 0.22 μm bottle top filter. Solution B is made by dissolving 0.3 g D(+)-glucose in 100 mL of solution A (16.7 mM final concentration). Solution C is made by mixing 10 mL of solution B and 90 mL of solution A.

Two 60 mm sterile petri dishes are pre-filled with 10 mL of solution C. Approximately 100 islets are transferred from the culture flask with less than 100 medium into one dish, then both dishes are incubated for 30 minutes at 37° C. At the end of the incubation period, the dish containing the islets is swirled to center the islets and the cells are transferred to the second dish in <50 μL of culture medium. Five Falcon 6 mL polypropylene tubes are labeled and pre-filled with 0.5 mL of solution C and five tubes are labeled and pre-filled with 0.5 mL of solution B. Five islets are hand-picked using a P20 micropipettor set to 5 μL and transferred into a pre-filled Falcon polypropylene tube. This process is repeated for each tube. The tubes are incubated without the cap in a 37° C. shaking water bath for 1 hour. After incubation, each tube is vortexed very gently for less than a second and centrifuged at 1400×g for 3 minutes. Two hundred μL of the supernatant are removed from each tube and stored in an identically labeled 1.5 mL microcentrifuge tube at 4° C. Immunoreactive insulin (IRI) is measured by an ELISA assay (Human Insulin EIA, Mercodia, Inc., Metuchen, N.J.).

The pelleted islets are washed once with 4 mL ddH₂O and immediately centrifuged at 1400×g for 3 minutes. The supernatant is aspirated and the pellet is dried completely using a Speed Vac.

To determine the DNA content, the pelleted cells are lysed with 500 μL of AT-Extraction solution (AT), which is prepared by mixing 33.33 mL of 1N ammonium hydroxide, 1 mL of Triton X-100, and 465.7 mL of ddH₂O. Each sample is sonicated for 10 seconds in ice water.

One hundred μL of AT solution are added to the first 8 wells of a 96-well microtiter plate and 100 μL of a dsDNA standard (2 μg/mL calf thymus DNA in TE buffer (10 mM Tris-HCl, 1 mM EDTA, pH to 7.5)) are placed in the first well of the 96-well plate (final concentration is 1 μg/mL). Seven 1:2 dilutions are performed with 100 to produce standards of 1, 0.5, 0.25, 0.125, 0.0625, 0.0313, 0.0156 and 0 μg/mL.

After adding 100 μL of sample to the remaining wells, 100 μL of PicoGreen (freshly prepared by mixing 0.5 μL PicoGreen and 99.5 μL TE Buffer per well) are added to each sample and standard. The plate is incubated for 15 minutes in the dark at room temperature and read on a fluorometer with an excitation of 480 nm and an emission of 520 nm. Released insulin content is calculated (from ELISA) and corrected for DNA concentration. Glucose-stimulated insulin release is expressed as μU IRI/ng DNA/60 min. A stimulation index is calculated by dividing insulin release at 16.7 mM glucose by insulin release at 1.7 mM glucose.

Viability: A fluorescent dye inclusion/exclusion assay is employed to assess metabolic activity and membrane integrity. London et al., Hormone & Metabolic Research—Supplement 1990; 25:82-87. Representative aliquots of 50 to 100 freshly isolated or cultured islets are transferred into a 1.5 mL microcentrifuge tube, touch spun (speed brought up to 1000 rpm and turned off immediately), and the supernatant discarded. The pelleted islets are resuspended in 0.67 μM fluorescein diacetate and 4 propidium iodide (PI), in a total volume 500 μL. Each tube is laid on its side, incubated in the dark at room temperature for 30 minutes without disturbing the tube. After the incubation, the islets are touch spun and the supernatant is removed. The pellet is resuspended in 405 μL of PBS. One hundred μL aliquots of the resuspended islets are transferred to different wells of a microtiter plate. Four 100 μL aliquots of PBS also are transferred to 4 wells of the microtiter plate. The plate is read on a fluorometer with an excitation/emission of 485/530 for fluorescein diacetate and 530/645 for PI. The percentage of viable islet cells per each of 50 consecutive islets is determined using the following formula, where X1=fluorescence of live cells under 485/530 filter for each well; X2=fluorescence of dead cells under 530/645 filter for each well:

$$\frac{( X 1 - {PBS}_{485 / 530} )}{( X 1 - {PBS}_{485 / 530} ) + ( X 2 - {PBS}_{530 / 645} )}$$

The mean and standard deviation also are calculated.

Adventitious Agent Testing: Samples are tested for aerobic and anaerobic organisms and fungi, and mycoplasma using known techniques. Islet cells suitable for transplantation are negative for each organism.

Endotoxin: One mL samples from the final supernatant before transplant are assayed using the Kinetic-QCL™ Test System (BioWhittaker, Walkersville, Md., cat #50-650U) for the presence of endotoxin. This chromogenic assay tests samples against a standard curve (0.005 to 50 EU/mL) with positive and negative controls. The standard curve is prepared using solutions from the Kinetic-QCL kit. Five endotoxin-free glass tubes are labeled with 50 EU/mL, 5 EU/mL, 0.5 EU/mL, 0.05 EU/mL, and 0.005 EU/mL. The 50 EU/mL standard is prepared by adding the amount of LAL Reagent Water specified on the Certificate of Analysis to the tube and vortexing at high speed for 5 minutes. The stock endotoxin solution is warmed to room temperature and vigorously vortexed again for 5 minutes. The 5 EU standard is prepared by adding 0.1 mL of the 50 EU/mL endotoxin stock into 0.9 mL of LAL Reagent Water. The 0.5 EU/mL endotoxin standard is prepared by transferring 0.1 mL of the 5 EU/mL endotoxin standard into 0.9 mL of LAL Reagent Water. The 0.05 EU/mL endotoxin standard is prepared by transferring 0.1 mL of the 0.5 EU/mL endotoxin standard into 0.9 mL of LAL Reagent Water. The 0.005 EU/mL endotoxin standard is prepared by transferring 0.1 mL of the 0.05 EU/mL endotoxin standard into 0.9 mL of LAL Reagent Water. Each of the solutions is vigorously vortexed for at least 1 minute before proceeding.

A BioTek ELX808 incubating plate reader is used to perform the assay. In the RUN mode, the specific test to be run is selected and the appropriate information concerning reagents and sample identification is entered as requested on the plate reader display. One hundred μL of each of the LAL Reagent Water blank, standards, product samples, positive product controls and sample control are dispensed into the appropriate wells of the microtiter plate.

The product samples, positive product controls and sample control are prepared as follows. If there is product inhibition from the yellow color of the transplant media, both undiluted and diluted (1:10) product samples are assayed. The undiluted sample is plated at 100 μL as stated above, however, the diluted sample is plated by first adding 90 μL of LAL Reagent Water to the appropriate well followed by 10 μL of product sample.

Undiluted and diluted samples of the transplant media alone serve as sample controls. Positive product controls are prepared using both undiluted and diluted product samples. For the undiluted positive product control, 90 μL of product sample and 10 μL of the 5 EU/mL endotoxin solution are plated. For the diluted positive product control, 80 μL of the LAL Reagent Water, 10 μL of the product sample, and 10 μL of the 5 EU/mL endotoxin solution are plated. Each well should contain a 0.5 EU/mL solution.

The filled plate is placed in the BioTek ELX808 plate reader and the UP directional key is pressed to position the plate in the incubation chamber. The assay is performed with the microtiter plate cover removed.

The plates are pre-incubated for ≧10 minutes. Near the end of the pre-incubation period, the Kinetic-QCL reagent is reconstituted by adding 2.6 mL of the LAL Reagent Water and mixing gently. The reconstituted Kinetic-QCL reagent is pooled into a reagent reservoir and mixed gently by rocking the reservoir from side to side. The DOWN directional key is pressed to remove the microtiter plate from the incubation chamber.

Using an eight channel pipettor, 100 μL, of the Kinetic-QCL reagent are dispensed into all wells, while avoiding causing bubbles, of the plate beginning with the first column (A1-H1) and proceeding in sequence to the last column used. The ENTER key is pressed to initiate the assay. The plate reader is continuously monitoring the absorbance at 405 nm of each well of the microtiter plate. Using the initial absorbance reading of each well as its own blank, the reader determines the time required for the absorbance to increase 0.200 absorbance units. This time is termed Reaction Time. The WinKQCL™ software automatically performs a log/log linear correlation of the Reaction Time of each standard with its corresponding endotoxin concentration. If the absolute value of the correlation coefficient (r) is ≧0.980, a polynomial model can be used to construct a standard curve and then to predict endotoxin concentrations of test samples. Islet products are released if the total endotoxin dose is less than or equal to 5 EU/kg body weight.

Diabetic Nude Mouse Bioassay: Athymic nude mice (National Institutes of Health, Bethesda, Md.) are rendered diabetic by an intravenous injection of streptozotocin (240 mg/kg). Following verification of diabetes (blood glucose >350 mg/dL×2 days), nude mice receive a renal subcapsular transplant of 750, 1000, or 2000 IE. Mice are bled every day posttransplant for the first 7 days and every Monday, Wednesday, and Friday for the following 3 weeks. A cure is defined as 3 consecutive daily blood glucose levels <200 mg/dL. If mice are still cured at one month posttransplant, the kidney that received the islets is removed for histological analysis and daily blood glucose levels are measured until diabetes returns (>300 mg/dL). Once diabetes is re-established, the mouse is euthanized. At 30 days posttransplant, mice are fasted for 24 hours prior to intraperitoneal injection of 1 g/kg body weight glucose in saline and plasma glucose values are obtained at 0, 30, and 120 minutes after glucose injection. The area-under-the-curve is used for the analysis.

### Example 3

### Diabetes Reversal after Single-Donor, Marginal-Dose Islet Transplants with Potent Induction Therapy and Calcineurin Inhibitor Minimization

This example assesses the safety of an islet transplant protocol in type 1 diabetic recipients, as well as the proportion of islet transplant recipients who achieve insulin independence in the first year after single-donor islet transplants. In brief, eight study participants with type 1 diabetes (T1D) accompanied by recurrent hypoglycemia unawareness or progressive secondary complications were enrolled in a prospective, single-center, 1-year follow-up trial. Study participants underwent a primary islet allotransplant with 7,271±1,035 islet equivalents/kg prepared from a single cadaver donor pancreas. Induction immunosuppression was with antithymocyte globulin, daclizumab, and etanercept. Maintenance immunosuppression consisted of mycophenolate mofetil, sirolimus, and no or low-dose tacrolimus.

Safety was assessed by monitoring the severity and duration of adverse events. Efficacy was assessed by studying the recipients' insulin requirements, C-peptide levels, oral and intravenous glucose tolerance results, intravenous arginine stimulation responses, and HbA_{1c} levels.

No serious, unexpected, or procedure- or immunosuppression related adverse events were observed. All 8 recipients achieved insulin independence and freedom from hypoglycemia. Five have remained insulin-independent for >1 year; their engraftment index was 150±29×10⁻⁶ ng·kg/mL. Graft failure in 3 recipients was preceded by subtherapeutic sirolimus exposure in the absence of measurable tacrolimus trough levels.

The tested protocol restored insulin independence and protected against hypoglycemia after single-donor, marginal-dose islet transplants in 8 of 8 recipients. Without being bound to a particular mechanism, the results may be related to improved islet engraftment secondary to peritransplant anti-thymocyte globulin and etanercept administration.

### Materials and Methods

Study Design. Recipients were defined as insulin-independent if they maintained fasting blood glucose levels below 126 mg/dL and 2-hour postprandial levels below 180 mg/dL after insulin discontinuation. The study protocol was approved by the local Institutional Review Board, and written informed consent was obtained from all participants.

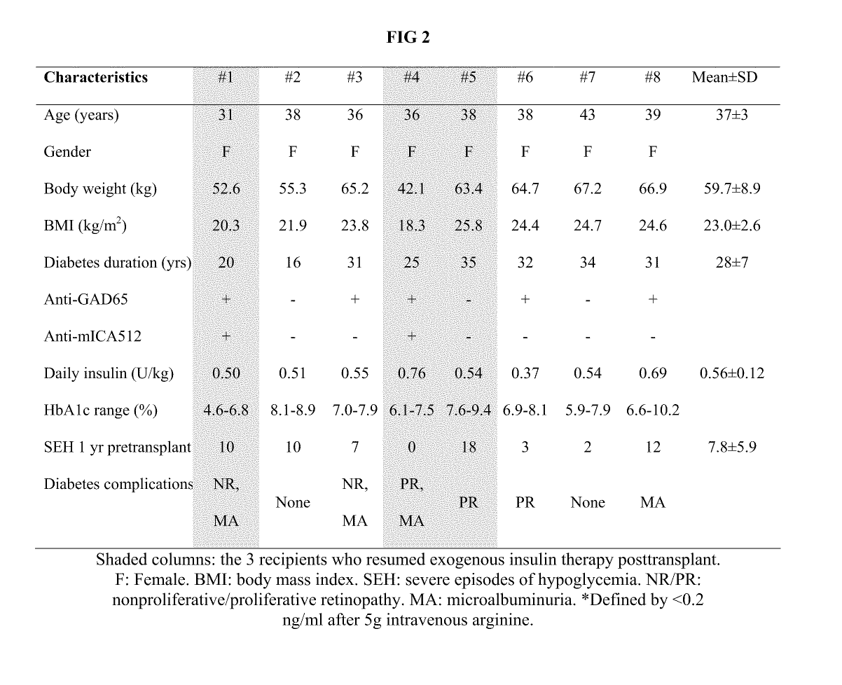

Patient Eligibility. Patients aged 18 years and older were eligible if they had C-peptide negative (<0.2 ng/mL after 5 g intravenous arginine) T1D for >5 years that was complicated by hypoglycemia unawareness (≧4 “reduced” responses in the hypoglycemia questionnaire developed by Clarke et al. (Diab. Care, 1995; 18:517-528), by metabolic lability/instability ≧2 episodes of severe hypoglycemia (The DCCT Research Group, Am. J. Med. 1991; 90(4):450-459) or ≧2 hospital admissions for ketoacidosis over the last year), or by progressive secondary complications despite intensive efforts made in close cooperation with their diabetes care team were eligible to participate. Progressive secondary complications were defined by (i) a new diagnosis during the last year by an ophthalmologist of proliferative retinopathy or clinically significant macular edema or therapy with photocoagulation; (ii) urinary albumin excretion rate >300 mg/day but proteinuria <3 g/day; or (iii) symptomatic autonomic neuropathy (as defined by postural hypotension in the setting of euvolemia, gastroparesis or diarrhea attributed to diabetic neuropathy, or neuropathic bladder as diagnosed by an urologist). Patients were excluded from the study if their renal function was abnormal (creatinine clearance <60 mL/min/1.73 m²) or if they had previously undergone an islet transplant. A total of 8 patients (coincidentally all female) were enrolled. Detailed recipient characteristics are shown in FIG. 2.

Pancreas Procurement and Preservation. Eighteen consecutive donor pancreases were procured from cadaver donors <50 years old with a BMI ≧27 kg/m²; the pancreases were preserved for ≦8 hours using the two-layer method (Tanioka et al., Surgery 1997; 122(2):435-441). See also description in Example 1. Donor exclusion criteria included a history of diabetes; risk factors or positive test results for HIV, hepatitis B, or hepatitis C; systemic infection; any pancreatic trauma; and any extracranial tumors. ABO compatibility and a negative serum cross-match for T cells were required, but HLA antigen matching was not required. Transplanted islets were prepared from 8 of the 18 donor pancreases (mean age of 39±12 years); the mean cold storage time was 6.6±1.7 hours. All donors had at least 1 serum glucose reading <200 mg/dL during their final hospitalization. The mean number of matched antigens at the HLA-A and -B loci was 1±0.5; and at the HLA-DR locus was 0.1±0.3. Detailed donor and graft characteristics are shown in Table 3.

TABLE 3

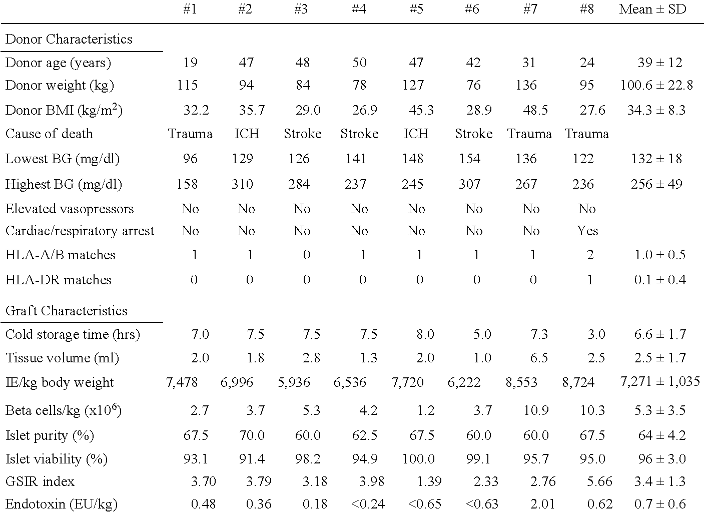

Shaded columns: the 3 recipients who resumed exogenous insulin therapy posttransplant. BG: blood glucose. ICH: intracerebral hemorrhage. IE: islet equivalents. GSIR: glucose-stimulated insulin release. PP: Pancreatic polypeptide. Elevated vasopressors: dopamine >20 μg/kg/min and/or norepinephrine at any dose. Islet composition is given per kilogram recipient body weight. Calculation of beta cells: derived from the total DNA content and the percentage of beta cells in the graft. Viability: based on analysis of islet aliquots stained with fluorescein diacetate and propidium iodide. GSIR index: amount of insulin released in vitro at high glucose (16.7 mM) divided by amount of insulin released at low glucose (1.67 mM) (each corrected for DNA content).

Islet Preparation. Islets were isolated as described in Example 1. Briefly, preserved pancreases were perfused with cold Liberase. Islets were isolated by the automated method, purified with continuous iodixanol density gradients in a Cobe 2991 cell separator, cultured free-floating in supplemented CMRL 1066 for 1 day at 37° C. and 1 day at 22° C. (Jahr et al., Cell Transplant., 2002; 11(6):513-518), and subjected to quality control (Ricordi et al., Acta Diabetol. Lat. 1990; 27:185-195). Independent quality assurance staff released islet products for transplant if they had verified documentation of islet enumerations of 5,000 to 20,000 IE per kg recipient body weight, an in-vitro glucose-stimulated insulin release index >1, viability ≧70%, total tissue pellet volume ≦10 cc, negative Gram stain, and endotoxin content ≦5 EU/kg. Specimens also were processed for aerobic, anaerobic, and fungal culture; mycoplasma; insulin; and DNA content for post-release information (see Example 2 for detailed methodology). Of the 18 consecutive cadaver donor pancreases processed for this study, preparations from 10 of them were not transplanted because of low islet yield. The average islet yield of 7 of those 10 preparations was 301,428±59,780 IE and would have met release criteria for a second islet transplant in recipients with partial function after their first transplant.

Transplant Procedure. After establishing access to the portal vein via minilaparotomy or percutaneous transhepatic portal venous catheterization, 7,271±1,035 IE per kg recipient body weight were infused, by gravity, along with 70 units/kg heparin on day 0. Prophylactic anticoagulation was continued with intravenous heparin (target partial thromboplastin time: 50 to 60 seconds) for 48 hours, followed by enoxaparin (30 mg subcutaneously twice daily) through day +7.

Immunosuppression. Induction immunosuppression, initiated on day −2, consisted of rabbit antithymocyte globulin (RATG; 0.5 mg/kg, day −2; 1.0 mg/kg, day −1; 1.5 mg/kg, days 0 through +2) (Agha et al., Transplantation 2002; 73(3):473-475), methylprednisolone (on day −2 only, 2 mg/kg), daclizumab (5 doses of 1 mg/kg every 2 weeks starting on day 0), and etanercept (50 mg intravenously, 1 hour pretransplant, followed by 25 mg subcutaneously on days +3, +7, and +10). Premedication for RATG included acetaminophen (650 mg) and diphenhydramine (50 mg) orally 30 minutes prior to and halfway through RATG infusion, as well as pentoxifylline (400 mg) orally three times daily (which was extended though day +7 posttransplant).

Maintenance immunosuppression was initiated with sirolimus (0.2 mg/kg starting on day −2, followed by 0.1 mg/kg daily; target whole blood trough levels 5 to 15 ng/mL, as tolerated), and reduced-dose tacrolimus (0.015 mg/kg twice daily, starting on day +1; target whole blood trough levels 3 to 6 ng/mL). At 1 month posttransplant, tacrolimus was gradually replaced with mycophenolate mofetil (MMF; 750 to 1000 mg, twice daily); tacrolimus was either discontinued or dosed to a target trough level of <3 ng/mL. If target levels of sirolimus could not be achieved or maintained, however, tacrolimus (target level, 3 to 6 ng/mL) was continued.

Concomitant Therapy. Standard antimicrobial prophylaxis was administered. Glycemic control was achieved with intravenous insulin through day +2 and subcutaneous insulin for at least 3 additional weeks.

Safety Assessments. Recipients underwent clinical, laboratory, and diagnostic safety assessments daily during their transplant hospitalization and at each of their 19 posttransplant study visits. Portal pressure was monitored during the transplant procedure. On days +1 and +7, ultrasonographic examination was done of the right upper quadrant including Doppler examination of the portal venous system. The severity and duration of any adverse events were monitored and recorded. Laboratory tests assessed hematology, chemistry, liver transaminases, lipids, standard creatinine clearance, and urinary albumin excretion. Adverse events were graded per the National Cancer Institute's Common Toxicity Criteria, Version 2.0.

Efficacy Assessments. Recipients recorded their insulin requirements and ≧5 self measured blood glucose concentrations daily. Throughout the study at each study visit before and after their transplant, recipients were asked about episodes of hypoglycemia. Before their transplant and then monthly posttransplant, basal C-peptide levels were determined by radioimmunoassay (reference range, 0.8 to 4 ng/mL) and hemoglobin A_{1c} (HbA_{1c}) levels were determined by high performance liquid chromatography (reference range, 4.3 to 6.0%). Recipients also underwent oral and intravenous glucose tolerance testing (OGTT and IVGTT) and intravenous arginine stimulation. The acute C-peptide response to arginine (ACR-Arg) was defined as the mean of the three highest insulin or C-peptide values between 2 and 5 minutes after the start of the arginine infusion, with the mean of the −10 and 0 values subtracted (Teuscher et al., Diabetes 1998; 47(3):324-330). Full islet graft function was defined as insulin independence and HbA_{1c}≦6.0%. Partial islet graft function was defined by insulin dependence, basal or arginine stimulated C-peptide levels of ≧0.5 ng/mL, and HbA_{1c}≦6.0%. Islet graft loss was defined by the absence of basal and arginine-stimulated C-peptide levels or by patient death.

Autoantibody Measurements. Anti-GAD65 antibody, anti-ICA512, and anti-insulin antibody titers were measured with radiobinding assays (Verge et al., Diabetes 1998; 47(12):1857-1866).

Statistical Analysis. Data are presented as mean±standard deviation unless otherwise stated. Comparisons were performed using the two-tailed Student's t-test.

### Results

Adverse Events. None of the 8 recipients experienced a serious, protocol-related, or unexpected adverse event. Transient elevation of liver enzymes was noted in 7 recipients after the transplant procedure. Transient diarrhea was noted in 6 recipients after they began MMF therapy, but it resolved or improved with dose reduction. Mild (5% to 10%, n=5) to moderate (10% to 20%, n=3) weight loss was noted in all 8 recipients. Hematologic adverse events included transient anemia, requiring transfusion without evidence of hemorrhage in 2 recipients (associated with hysterectomy in 1 and with RATG, sirolimus, and hemodilution in the other), and transient neutropenia, requiring short-term (<1 week) G-CSF in 5 recipients (3 received 1 dose) without evidence of infection. Shortly after receiving ATG, 6 recipients developed lymphopenia; their mean lymphocyte count at 1 year posttransplant was 0.4±0.2×10⁹/L. All 8 recipients experienced intermittent oral aphthous ulcers during the study. One recipient had a decrease in creatinine clearance, possibly related to a sampling error, but at the study close-out visit, serum creatinine was 0.6 mg/dL. Another recipient showed a 30% decrease in creatinine clearance with an unchanged serum creatinine (0.9 mg/dL). Serum creatinine and creatinine clearance remained unchanged (≦1 mg/dL) in the other 6 recipients. No increase in urine albumin excretion was noted in any recipient. Serum LDL increased from <100 in 1 recipient and from 100-130 mg/dL in another to >130 mg/dL. None of the 8 recipients experienced a procedural complication related to the islet transplant. More specifically, none experienced hemorrhage or portal vein thrombosis. Portal pressure increased from 9 to 16 cm H₂O in 1 recipient, but no significant increase was noted in 7 other recipients. Average portal pressure was 9±5 cm both before and after islet infusion.

Posttransplant Islet Function. All 8 recipients became insulin-independent, with normal HbA_{1c} levels and freedom from hypoglycemia. The time to insulin independence ranged from 23 to 122 days. Of the 8 recipients, 5 have remained insulin-independent for >1 year and 3 were insulin-independent for 121, 76, and 7 days (see Table 4). For 4 of these 5 recipients, oral glucose tolerance testing at ≧180 days posttransplant revealed normal 2-hour plasma glucose levels. On day +180 or later, acute insulin responses to intravenous arginine in the 5 recipients with sustained insulin independence averaged 15.5±3.7 μU/mL (53±13% of controls); and to glucose, 16.7±5.5 μU/mL (30±10% of controls). Their acute C-peptide responses to intravenous arginine averaged 1.07±0.15 ng/mL (59±8% of controls); and to glucose, 1.23±0.46 ng/mL (40±15% of controls). Please see Table 4 for additional metabolic monitoring information.

The 5 recipients received daily MMF doses of 1.5 to 2.0 g. They either (a) achieved and maintained sirolimus trough levels >9 ng/mL, with tacrolimus trough levels of 0 to <3 ng/mL, or (b) in the absence of target sirolimus trough levels, tacrolimus trough levels of 3 to 6 ng/mL. The 3 recipients who resumed exogenous insulin therapy had received ≧1.5 g/day of MMF but had subtherapeutic sirolimus trough levels (<9 ng/mL) in the absence of measurable tacrolimus trough levels (<3 ng/mL). Please see Table 5 for immunosuppression.

TABLE 4
Islet graft function for each of 8 islet recipients

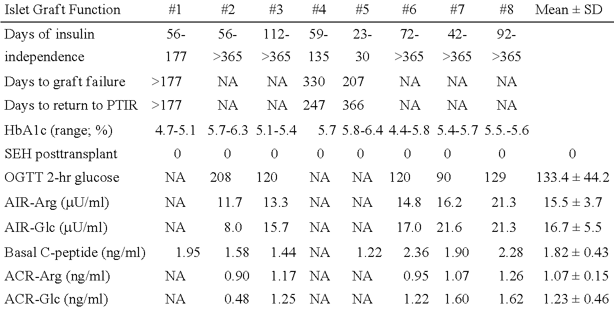

Shaded columns: the 3 subjects who resumed exogenous insulin therapy posttransplant. PTIR: pretransplant insulin requirements. HbA1c: Hemoglobin A1c. NA: not applicable. OGTT: oral glucose tolerance test. AIR/ACR-Arg/Glc: acute insulin/C-peptide response to intravenous arginine/glucose (average of the 3 peak levels minus average pre-challenge level). Normal, non-diabetic controls showed an average AIR-Glc of 56.1 ± 8.4 μU/ml, ACR-Glc of 1.04 ± 0.17 ng/ml, AIR-Arg of 29.1 ± 5.3 μU/ml, and ACR-Arg of 0.60 ± 0.07 ng/ml.

TABLE 5
Drug exposure and portal venous access route for each of 8 islet transplant

| | #1 | #2 | #3 | #4 | #5 | #6 | #7 | #8 |
|---|---|---|---|---|---|---|---|---|
| Drug Exposure* | | | | | | | | |
| Mycophenolate (g/day) | 1.5 | 2.0 | 1.5-2.0 | 1.5-2.0 | 1.5 | 1.0-1.5 | 1.5-2.0 | 1.5 |
| Sirolimus (ng/ml) | 4-8 | >9 | 2-3 | 7-9 | 4-9 | 6-8 | >9 | >9 |
| Tacrolimus (ng/ml) | 0 | 0 | 4-5 | 0-<3 | 0-<3 | 4-6 | <3 | 3 |
| Portal Vein Access | | | | | | | | |
| Minilaparotomy | ✓ | ✓ | ✓ | ✓ | ✓ | | | |
| Percutaneous transhepatic | | | | | | ✓ | ✓ | ✓ |

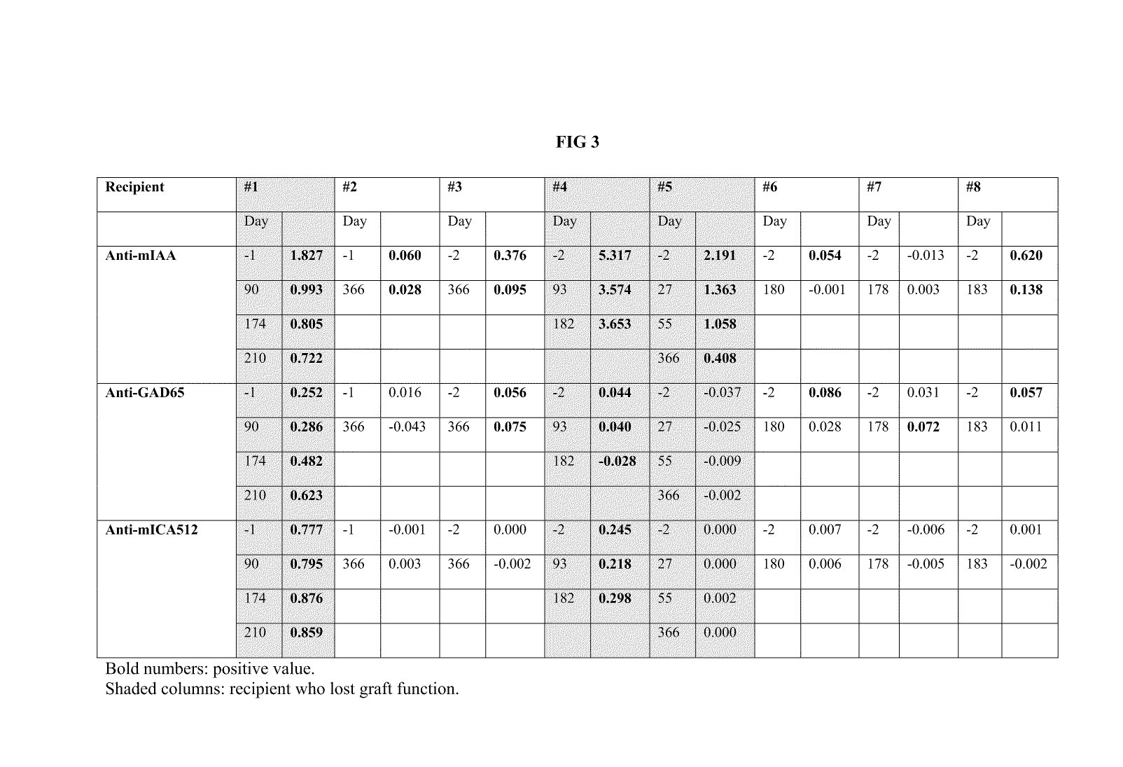

Autoantibodies. As shown in FIG. 3, of the 3 subjects with graft failure, 2 were positive for both anti-GAD and anti-ICA512 in the pretransplant period. In contrast, none of the 5 who remained insulin-independent was positive for anti-GAD and anti-ICA512.

Alloantibodies. Graft failure was followed by allosensitization in 2 recipients.

The results mark a distinct advance in islet transplant efficiency. Not only was insulin independence achieved with islets from only 1 donor pancreas (as compared with 2 to 4 in the Edmonton trial (Shapiro et al., New Engl. J. Med. 2000; 343(4):230-238)), superior glycemic control was achieved (as evidenced by normal OGTT results in 4 of 5 recipients with sustained insulin independence) with significantly fewer islets (7,271±1,035 versus 11,547±1,604 IE/kg; p<0.0001).

### Example 4

### Transplantation of Cultured Islets from Two-Layer Preserved Pancreases in T1D with Anti-CD3 Antibody

This example describes a set of experiments to determine whether or not optimizing pancreas preservation, islet processing, and induction immunosuppression would facilitate sustained diabetes reversal after single-donor islet transplants. Islets were isolated from two-layer preserved pancreata, purified, cultured for 2 days; and transplanted into six C-peptide-negative, nonuremic, T1D patients with hypoglycemia unawareness. Induction immunosuppression, which began 2 days pretransplant, included the Fc receptor nonbonding humanized anti-CD3 monoclonal antibody hOKT3γ1 (Ala-Ala) and sirolimus. Immunosuppression was maintained with sirolimus and reduced-dose tacrolimus. Of the six recipients, four achieved and maintained insulin independence with normal HbA_{1c} levels and freedom from hypoglycemia; one had partial islet graft function; and one lost islet graft function 2 weeks posttransplant. The four insulin-independent patients showed prolonged CD4⁺ T-cell lymphocytopenia; invented CD4:CD8 ratios; and increases in the percentage of CD4⁺CD25⁺ T cells. These cells suppressed the in-vitro proliferative response to donor cells and, to a lesser extent, to third-party cells. Severe adverse events were limited to a transient rash in one recipient and to temporary neutropenia in three. The results suggest that a combination of maximized viable islet yield, pretransplant islet culture, and preemptive immunosuppression can result in successful single-donor islet transplants.

### Materials and Methods

Study design. A single-center, prospective, open-label pilot study was performed in T1D recipients receiving their first islet-only transplant. Islets were prepared from a single-donor pancreas (preserved with the two-layer preservation method) and cultured for 2 days pretransplant. All participants received hOKT3γ1 (Ala-Ala) and sirolimus induction therapy initiated on day −2 pretransplant, followed by maintenance immunosuppression with sirolimus and tacrolimus. Participants were monitored for 1 year after their islet transplant. The study protocol was approved by the institutional review board at the University of Minnesota, and all participants gave written informed consent.

Study eligibility. Individuals who were at least 18 years old with T1D mellitus for >5 years were eligible for the study if they met at least 1 of the following criteria: reduced awareness of hypoglycemia (≧4 “reduced” responses in the hypoglycemia questionnaire developed by Clarke et al., 1995 supra); metabolic lability or instability, characterized by 2 or more episodes of severe hypoglycemia (an event with symptoms consistent with hypoglycemia in which the assistance of another person is required and which is associated with a blood glucose below 50 mg/dL or prompt recovery after oral carbohydrate, intravenous glucose, or glucagon administration) or 2 or more hospital admissions for diabetic ketoacidosis over the previous year; or progressive secondary complications. Such complications could be defined by (i) a new diagnosis per an ophthalmologist, of proliferative retinopathy, by clinically significant macular edema, or by photocoagulation therapy during the last year; (ii) a urinary albumin excretion rate >300 mg/day but proteinuria <3 g/day; or (iii) symptomatic autonomic neuropathy. Individuals were excluded from our study if they had basal or stimulated serum C-peptide levels ≧0.2 ng/mL; HbA_{1c} levels >12%; body weight >70 kg. Other exclusion criteria include the presence or history of anti-HLA antibodies directed against >10% of a panel of lymphocyte donors (representing the pool or possible organ donors); insufficient cardiovascular reserve; creatinine clearance <60 mL/min/1.73 m²; portal hypertension; presence or history of liver disease; active peptic ulcer disease; severe unremitting diarrhea; active infections; serologic evidence of infection with HIV, hepatitis B, hepatitis C; negative Epstein-Barr virus serologic results; and a history of malignancy within the past 10 years (other than adequately treated basal or squamous cell carcinoma of the skin). Eligible individuals were subsequently reviewed by an independent endocrinologist to confirm or contest their eligibility. In addition, healthy and type 1 diabetic control subjects provided, with consent, blood samples for the determination of the frequency of CD4⁺CD25⁺ T cells in peripheral blood.

Immunosuppression. Study participants received 12 doses of intravenous hOKT3γ1 (Ala-Ala), daily, beginning 2 days before their islet allograft transplant and continuing through day +9 posttransplant. On day −2 pretransplant, they received a 1-mg dose of hOKT3γ1 (Ala-Ala); on day −1, a 2-mg dose. On days 0 through +9 posttransplant, they received a daily 4-mg dose. Dosing was increased by 25% to 50% if recipients did not achieve ≧80% CD3 coating after the first 5 infusions of hOKT3γ₁ (Ala-Ala). The doses were based on those previously used to treat kidney graft rejection (Woddle et al., Transplantation 1999; 68:608-616). Premedication was with acetaminophen, diphenhydramine, and pentoxifylline. Sirolimus (Rapamune, Wyeth-Ayerst) was initiated orally at a dose of 0.2 mg/kg on day −2, followed by 0.1 mg/kg daily; the dose then was titrated and adjusted to achieve a target whole blood trough level of 5 to 10 ng/mL. Mean trough concentration prior to transplant on day 0 was 6.1±1.7 ng/mL and on day +2 was 6.7±1.9 ng/mL. Reduced-dose tacrolimus (Prograf, Fujisawa) was administered orally to all islet recipients beginning on day +8 posttransplant at 0.015 mg/kg 2 times per day; the dose was then adjusted to achieve a target whole blood trough level of 3 to 6 ng/mL (Shapiro et al., New Engl. J. Med. 2000; 343:230-238).

Monitoring hOKT3γ1 (Ala-Ala). Flow cytometry was used to enumerate CD2⁺, CD4⁺, CD8⁺, CD25⁺, and CD69⁺ T cells. Coating and modulation of the CD3 molecule was determined on PBLs, serum levels of hOKT3γ1 (Ala-Ala), and serum cytokines, as previously described (Woodle et al., 1999 supra). Anti-idiotype antibodies were identified by ELISA, by the use of plate-bound OKT3, or by flow cytometry, in order to measure the blockade of binding of OKT3 fluorescein isothiocyanate to CD3 (Chatenoud, Transplant Proc. 1993; 25:68-73).

Proliferation assay. Peripheral blood T cells or CD4⁺ T cells were collected on days 651, 624, 617, 440, and 141 following hOKT3γ1 (Ala-Ala) treatment and subsequently isolated by negative selection with either CD4⁺ or T Cell Enrichment RosetteSep (Stem Cell Technologies). CD4⁺CD25⁺, CD4⁺CD25⁻ T-cell subsets were separated by magnetic beads (Miltenyi) or by sorting stained T cells (CD4-FITC, CD25-PE, CD45-APC) on a FACSVantage flow cytometer (Becton Dickinson Biosciences). Separated CD4⁺CD25⁻ or CD4⁺ T cells were labeled with CFSE. After washing, the subsets (5×10⁴) were cultured with or without the addition of titrated numbers of CD4⁺CD25⁺ T cells in 200 μl of X-VIVO-15 supplemented with L-glutamine (Biowhittaker), then, the subsets were stimulated with 4×10⁵ irradiated (3,000 Gy) donor or third-party splenocytes. After 6 to 7 days, CFSE-fluorescence of lymphocytes was analyzed on a FACSCalibur and the percentage of proliferative events of total CFSE-stained lymphocytes was determined. The percent reduction in proliferation to allogeneic antigen-presenting cells was calculated (1-[proliferative events in cultures with added CD4⁺CD25⁺ cells]/1−[proliferative events in cultures without added CD4⁺CD25⁺ cells]).

Autoantibody assay. Anti-GAD65 antibody, anti-ICA512, and anti-insulin antibody with radiobinding assays (Verge et al, 1996, supra).

Concomitant therapy. Standard antimicrobial prophylactic prophylaxis was administered. Heparin was co-infused intraportally with islets at a dose of 70 U/kg; anticoagulation prophylaxis was continued for the first 48 hours posttransplant with intravenous heparin and was targeted to partial thromboplastin time to 50 to 60 seconds. Enoxaparin (30 mg subcutaneously twice a day) was initiated after heparin discontinuation and continued through day +7 posttransplant. Aspirin (daily oral dose of 81 mg) was started on day +1 posttransplant. Insulin was administered posttransplant as needed to achieve and maintain normoglycemia. Recipients who were able to maintain fasting blood glucose levels below 126 mg/dL and 2-hour postprandial levels below 180 mg/dL after insulin discontinuation were considered insulin-independent.

Pancreas donor selection. Pancreases were obtained from brain-dead, heart-beating multi-organ donors who were between 15 to 50 years old. Exclusion criteria were the same as in Example 3. A negative serum cross-match for T cells and for ABO compatibility was required. HLA antigen matching was not required.

Pancreas procurement and preservation. The pancreas was removed after cold perfusion with UW solution (ViaSpan, DuPont Pharma) using standardized techniques developed for combined liver and pancreaticoduodenal procurement (Marsh et al., Surg. Gynecol. Obstet. 1989; 168:254-258). Cold storage preservation of each donor pancreas was performed using the two-layer method in a custom-made organ shipping container, in which 500 mL of UW solution was poured on top of 750 mL of perfluorodecalin (FluoroMed, L. P.). See Example 1. Medical-grade oxygen gas was administered through a filter to the perfluorodecalin solution at a rate of 75 mL/min for 40 minutes to achieve oxygen saturation. About 60% of the pancreas was submerged in perfluorodecalin. Cold storage time was less than 8 hours for all pancreases.

Islet preparation. Islet processing was performed in compliance with federal regulations. Islets were isolated, purified, and cultured as described in Examples 1 and 3. Fractions with islet purities (percentage of DTZ-positive cells) >10% were combined and cultured overnight at 37° C. and for an additional 24 to 48 hours at 22° C. as described in Example 1. The average recovery of islet equivalents after culture for the 6 transplanted islet products was 118±35% and insufficient recovery post-culture to permit transplantation was not observed. The increase in number of islet equivalents after culture is presumably due to the precision limit of assessing islet yield.

Islet quality control. Characterization and release testing of islet products were performed as described in Example 3. Of the last 60 consecutive islet preparations, only one was found to be contaminated with Candida glabrata; no other adventitious agents have been noted. In addition, 3 islet aliquots (750 IE; 1,000 IE; and 2,000 IE) of each islet preparation were transplanted into the renal subcapsular space of diabetic nude mice and tested for diabetes reversal by daily glucose monitoring.

Islet transplant procedure. Pretransplant, islets were suspended in CMRL-1066 supplemented with 2.5% human serum albumin and 25 mM HEPES, and with heparin added at 70 U/kg recipient body weight as described in Example 1. Recipients were sedated, local anesthesia was provided, and islets were infused over 15 to 60 minutes into a mesenteric or omental vein accessed by mini-laparotomy.

Safety assessments. Recipients underwent clinical, laboratory, and diagnostic safety assessments. Daily during their transplant hospitalization and at each of their 23 posttransplant study visits, the severity and duration of any adverse events were monitored and recorded. Laboratory tests included hematology, chemistry, liver transaminases, lipids, standard creatinine clearance, and urinary albumin excretion. Adverse events were graded based on the National Cancer Institute's Common Toxicity Criteria, Version 2.0.

Efficacy assessments. Efficacy assessments were performed as described in Example 3. Full islet graft function was defined as insulin independence and HbA_{1c ≦}6%. Partial islet graft function was defined by insulin dependence, basal or arginine-stimulated C-peptide levels of 0.5 ng/mL, and HbA_{1c}<7%. Islet graft loss was defined by the absence of basal and arginine-stimulated C-peptide levels or by patient death.

Statistical analysis. Data are presented as mean±standard deviation unless otherwise stated. Statistical analysis was not performed, given the low number of recipients.

### Results

Recipient characteristics. A total of 6 patients (Table 6) with T1D complicated by hypoglycemia unawareness underwent a single-donor islet transplant; 5 participated for the entire planned 1-year posttransplant follow-up period. (Recipient #4 declined further participation after day +93 posttransplant except for a blood sample for T-cell subsets on day 323.)

TABLE 6
Recipient characteristics

| Recipient Number | #1 | #2 | #3 | #4 | #5 | #6 |
|---|---|---|---|---|---|---|
| Age (years), gender (M/F) | 30, F | 35, F | 30, F | 46, F | 24, F | 35, M |
| Weight (kg), body mass index (kg/m²) | 63.0, 22.3 | 53.6, 22.3 | 64.9, 22.1 | 68.0, 25.4 | 69.0, 26.7 | 65.0, 22 |
| Diabetes duration (years) | 18 | 26 | 24 | 35 | 13 | 29 |
| Immediate pretransplant HbA1c (%) | 7.3 | 5.8 | 7.8 | 6.5 | 9.3 | 8.0 |
| Pretransplant HbA1c Range (%) | 7.4-7.9 | 5.4-6.4 | 7.0-8.9 | 6.3-7.4 | 6.6-11.7 | 7.6-8.6 |
| Insulin requirements (units/kg/day) | 0.4 | 0.8 | 0.4 | 0.6 | 0.8 | 0.5 |
| Number of severe hypoglycemic | 10 | 100 | 20 | 5 | 20 | 5 |
| episodes, 1 year pretransplant | | | | | | |
| Microvascular complications | None | Autonomic | None | Peripheral & | Peripheral & | Proliferative |
| | | neuropathy | | autonomic | autonomic | retinopathy, |
| | | | | neuropathy | neuropathy | autonomic |
| | | | | | | neuropathy |

Thirteen donor pancreases were processed in this study. Transplanted islets were prepared from 6 multi-organ donors (mean age of 25.8±9.4 years) (Table 7). All donors had at least 1 serum glucose reading <200 mg/dL during their hospitalization. The mean number of mismatched antigens at the HLA-A, -B, and -DR loci were 1.2±0.8, 1.7±0.5, and 1.5±0.5.

Islet graft characteristics also are shown in Table 7. The mean cold storage time of the 6 donor pancreases was 7.4±0.7 hours. The mean dose of transplanted islets was 10,302±2,594 IE per kg recipient body weight; the mean insulin content 0.86±0.46 units per kilogram body weight. The in vitro glucose-stimulated insulin secretory response stimulation index varied from 4.6 to 27.1, (mean of 13.5±8.2); the insulin release in response to 16.7 mM glucose varied from 0.17 to 0.97 μU/ng DNA. In addition to the 6 human transplants, islet aliquots (750, 1000, or 2000 IE) also were transplanted under the renal subcapsular space in streptozotocin-induced diabetic athymic nude mice. Aliquots from 4 of the 6 donors reversed diabetes within 4 days (Table 7).

TABLE 7
Donor and graft characteristics

| Recipient | Recipient | Recipient | Recipient | Recipient | Recipient |
|---|---|---|---|---|---|
| #1 | #2 | #3 | #4 | #5 | #6 |

Donor

| | | | | | | |
|---|---|---|---|---|---|---|
| Donor age (years) | 21 | 16 | 39 | 26 | 35 | 18 |
| Donor weight (kg) | 106 | 109 | 159 | 106 | 111 | 90 |
| Donor body mass index | 31.9 | 32.6 | 45.0 | 30.0 | 29.8 | 27.0 |
| (kg/m²) | | | | | | |
| Cause of death | Trauma | Non- | Cerebro- | Trauma | Trauma | Anoxia |
| | | traumatic | vascular | | | |
| Time in ICU (hours) | 27.5 | 36.0 | 19.0 | 138.0 | 6.0 | 280.0 |
| Minimum serum glucose | 128 | 162 | 106 | 137 | 135 | 64 |
| (mg/dL) | | | | | | |
| Maximum serum glucose | 236 | 392 | 130 | 258 | 214 | 424 |
| (mg/dL) | | | | | | |
| Elevated vasopressors^{†}, yes/no | No | No | No | Yes | Yes | No |
| Cardiac/respiratory arrests, | No | No | No | Yes | No | Yes |
| yes/no | | | | | | |

Graft

| Cold storage time (hours) | | 6.0 | 7.5 | 7.5 | 8.0 | 8.0 | 7.5 |
|---|---|---|---|---|---|---|---|
| IE/kg body weight | | 8,151 | 15,210 | 10,718 | 9,520 | 9,911 | 8,302 |
| IRI/kg body weight (U)^{‡} | | 1.00 | 1.00 | 0.74 | 0.32 | 0.47 | 1.60 |
| Stimulation index^{§} | | 17.6 | 14.4 | 7.0 | 4.6 | 10.5 | 27.1 |
| IRI_{16.7 mM glucose} (μU/ng DNA) | | 0.87 | 0.27 | 0.40 | 0.17 | 0.97 | 0.38 |
| Time to cure* | (2000 IE) | —^{♦} | 1 | 4 | 28 | No Cure | 4 |
| | (1000 IE) | 1 | No Cure | No Cure | No Cure | No Cure | 1 |
| | (750 IE) | 12 | No Cure | No Cure | No Cure | No Cure | 4 |

^{†}Elevated vasopressors: dopamine at ≧20 μg/kg/min or norepinephrine at any dose.
^{‡}IRI: immunoreactive insulin.
^{§}Stimulation index: ratio of in vitro insulin release in response to 16.7 mM glucose divided by response to 1.7 mM glucose.
*Time to cure: number of days to achieve stable normoglycemia (blood glucose <200 mg/dL) after transplantation of the indicated number of islet equivalents (IE) in streptozotocin-diabetic athymic nude mice.
^{♦}The mouse that received 2000 IE of the suspension prepared for recipient #1 died for technical reasons.

Posttransplant islet function. Of 6 patients, 4 (#1, #2, #3, and #6) became insulin-independent posttransplant and remained so throughout the posttransplant follow-up period of 365 days. The mean time to insulin independence was 35±7 days. One recipient (#4) showed a transient, roughly 50% reduction of daily insulin requirements until early islet allograft failure ensued on day +14 posttransplant. Recipient #5 showed partial islet graft function; exogenous insulin doses averaged 60% of pretransplant insulin requirements.

The 4 insulin-independent recipients showed significant improvement of glycemic control, with normal HbA_{1c} levels and no hypoglycemia throughout the posttransplant follow-up period (Table 8). The mean pretransplant HbA_{1c} level in all 6 recipients was 7.4±1.2%; the HbA_{1c} level in the 4 insulin-independent recipients was 5.9%, 5.3%, 5.4%, and 5.2% in quarters 1, 2, 3, and 4 posttransplant (vs. 7.5%, 5.8%, 7.3%, and 8.2% pretransplant). The mean plasma glucose level in the 4 insulin-independent participants 2 hours after administration of 75 g of oral glucose 180 days posttransplant was 113±37 mg/dl: a normal result in 3, but a response indicative of impaired glucose tolerance in 1. The acute C-peptide response to intravenous arginine in the 4 insulin-independent recipients ≧180 days posttransplant varied from 0.46 ng/mL to 1.08 ng/mL.

TABLE 8
Metabolic results.

| | | | | | | | Number of episodes of | | 2-hr OGTT | Basal | |
|---|---|---|---|---|---|---|---|---|---|---|---|
| | Time to insulin | Ave. HbA1c | | | | | severe hypoglycemia* | | plasma glucose | C-peptide | ACPR-Arg |
| | independence | (%) Pre- | Post-transplant | | | | Year | Year | (mg/dl) | (ng/mL) | (ng/mL) |
| Recipient | (days) | transplant | Q1 | Q2 | Q3 | Q4 | Pretx | Posttx | ≧180 days Posttx | | |
| #1 | 33 | 7.5 | 5.9 | 5.6 | 5.9 | 6.0 | 10 | 0 | 178 | 1.09-1.10 | 0.46-0.54 |
| #2 | 37 | 5.8 | 5.1 | 5.3 | 4.9 | 4.7 | 100 | 0 | 122 | 1.0-1.60 | 0.58-0.84 |
| #3 | 27 | 7.3 | 5.6 | 4.9 | 4.9 | 5.0 | 20 | 0 | 129 | 1.55-1.60 | 0.94-1.08 |
| #4 | NA | 6.5 | 6.7 | ND | ND | ND | 5 | 0 | ND | ND | ND |
| #5 | NA | 9.1 | 7.4 | 8.1 | 8.4 | 8.9 | 20 | 0 | ND | 0.22 | 0.09 |
| #6 | 43 | 8.2 | 6.8 | 5.3 | 5.8 | NA | 5 | 0 | 89 | 1.0-2.71 | 0.5-1.0 |

The number of episodes of severe hypoglycemia posttransplant is given for the duration of islet graft function.
*For the duration of graft function;
ND: not done; NA: not applicable or not achieved.

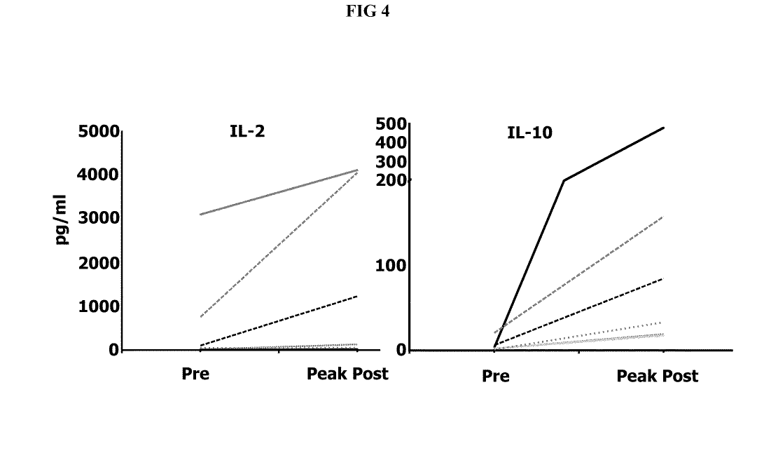

Monitoring of hOKT3γ1 (Ala-Ala). The mean total dose of hOKT3γ1 (Ala-Ala) was 45.7±10.5 mg (see Table 9). In participant #1, hOKT3γ1 (Ala-Ala) therapy was discontinued on day +6 posttransplant secondary to a generalized rash (initially presumed to be related to hOKT3γ1 [Ala-Ala]). In participants #4, #5, and #6, the dose of hOKT3γ1 (Ala-Ala) was increased (per the protocol) because 80% CD3 coating was not achieved after the first five infusions. The mean time to 80% CD3 coating was 7.3±2.9 days; the mean duration of CD3 coating 80%, 4.8±2.6 days. The mean peak hOKT3γ1 (Ala-Ala) level was 1196±821 ng/mL. The hOKT3γ1 (Ala-Ala) level was undetectable at 61±32 days after the last dose. Absence of anti-idiotype antibodies was verified by ELISA for all recipients treated with hOKT3γ1 (Ala-Ala). Serum IL-2 levels were substantially higher in recipients #4 and #5, as compared with the other 4, both before and after hOKT3γ1 (Ala-Ala) administration (FIG. 4). Serum IL-10 levels increased in all participants after hOKT3γ1 (Ala-Ala) administration, as compared with pre-dosing levels (FIG. 4). Mean serum TNF-α was 29.9±31.7 pg/mL prior to hOKT3γ1 (Ala-Ala) administration, and the mean peak afterwards was 267.6±120.3 pg/mL.

TABLE 9
Dosing and monitoring of hOKT3γ1 (Ala-Ala) immunotherapy

| | Total dose | Time to 80% | Duration >80% | Peak hOKT3γ1 | Days to negative |
|---|---|---|---|---|---|
| | hOKT3γ1 (Ala- | CD3 coating | CD3 coating | (Ala-Ala) level | hOKT3γ1 (Ala-Ala) |
| Subject | Ala) given (mg) | (# of days) | (Total # of days) | achieved (ng/mL) | level after last dose |
| #1 | 29 | 4.5 | 4 | 736 | 50 |
| #2 | 43 | 4.5 | 8 | 2761 | 33 |
| #3 | 43 | 5.5 | 8 | 1330 | 34 |
| #4 | 49 | 9.5 | 4 | 872 | 91 |
| #5 | 61 | 8.5 | 3 | 450 | 111 |
| #6 | 49 | 11.5 | 2 | 1028 | 47 |

Comparison of the 4 recipients (#1, #2, #3, and #6) who achieved insulin independence versus the 2 who did not (#4 and #5) revealed plausible technical and immunological explanations for the different outcomes. Recipients #4 and #5 received islets prepared from donors with elevated vasopressor requirements (dopamine >20 μg/kg/min and/or norepinephrine at any dose) and with increased serum creatinine levels (1.8 mg/dl, #4; 5.2 mg/dl, #5) suggestive of relevant hypotension. Recipient #4 received an islet graft containing 0.32 units insulin/kg recipient body weight, #5, 0.47. In contrast, the other 4 recipients received islet grafts containing 0.74-1.60 units insulin/kg recipient body weight. Notably, aliquots from all 4 islet grafts that restored insulin independence also promptly reversed diabetes in diabetic nude mice within the first 4 days posttransplant. However, aliquots from the 2 islet grafts that failed to restore insulin independence also failed to reverse diabetes in the mice in the first 4 weeks.

In addition to donor factors, the BMI (27 kg/m²), HbA1c (9.3%), and waist-to-hip ratio (0.96) of recipient #5 at the time of transplant were higher, as compared with the 4 insulin-independent recipients. Pretransplant, the insulin requirement for recipient #5 was 0.8 units/kg, higher than 4 of the 5 other recipients. Thus, recipient #5 showed clinical features of impaired insulin action (Williams et al., Diabetes 2000; 626-632), conceivably compromising the ability of an islet graft with marginal potency to restore insulin independence.

Peripheral T-cell subsets. Immunosuppression therapy resulted in profound depletion of peripheral CD2⁺, CD4⁺, and CD8⁺ T cells. Depletion of the CD2⁺ and CD8⁺ T cells was transient; however, CD4⁺ depletion persisted throughout the duration of the study, except in recipient #5 (FIG. 5). A persistently inverted CD4:CD8 ratio was achieved within the first month in recipients #1, 2, 3, and #6, but not in #5 (FIG. 5); samples from #4 were incomplete. About 3 weeks after completion of hOKT3γ1 (Ala-Ala) administration, the proportion and absolute number of CD4⁺ T cells that were also CD25⁺ began to increase in 3 of the 4 subjects who achieved insulin independence (FIG. 5). But in these 4 recipients, neither the proportion of CD4⁺ T cells that were also CD69⁺ nor the proportion of CD2⁺ lymphocytes that were also CD4⁺ increased during this period.

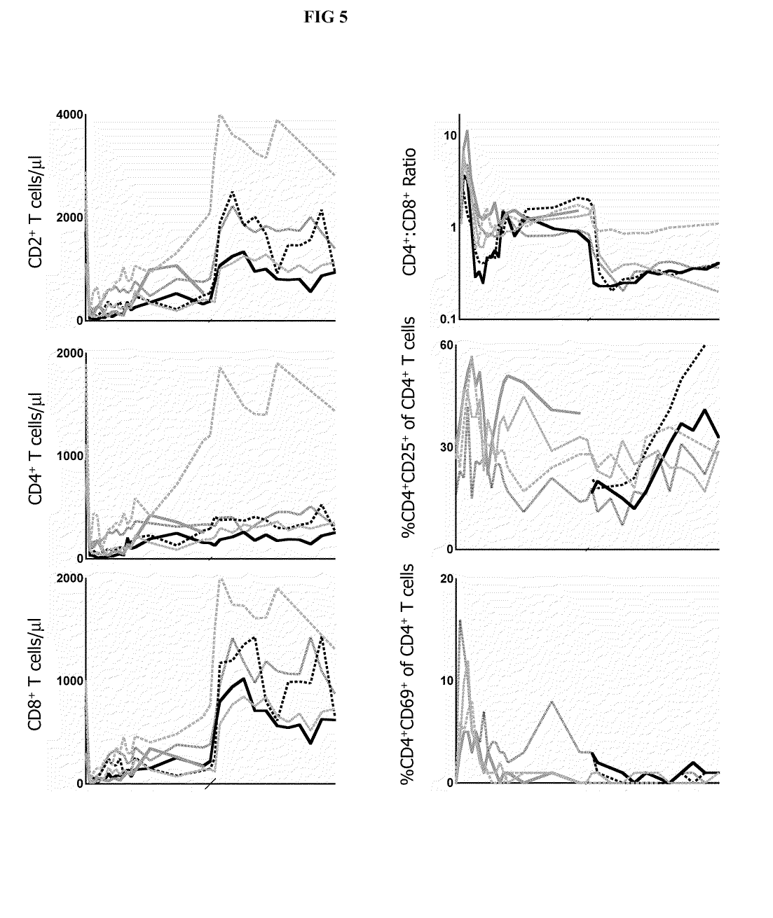

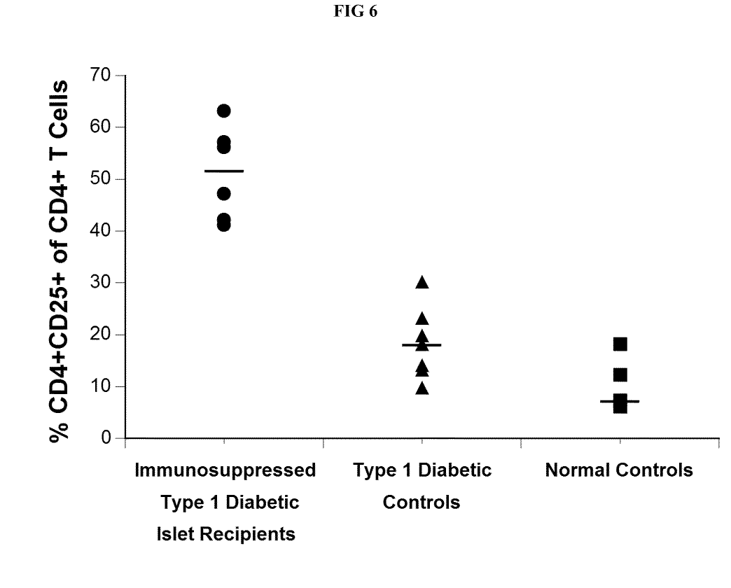

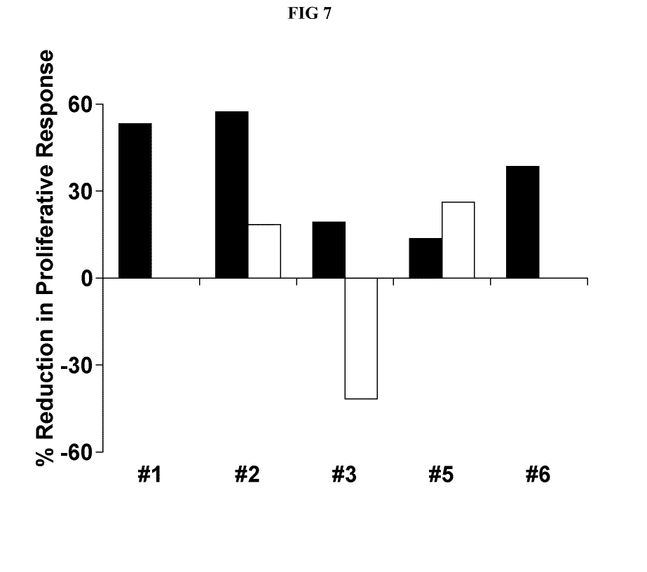

The percentage of CD4⁺CD25⁺ cells in healthy controls (n=5) ranged from 5.97% to 18%; in type 1 diabetic controls (n=7) from 9.6% to 23%, and 41% to 63% (all peak percentages) in our islet recipients (n=6) (FIG. 6). The regulatory function of CD4⁺CD25⁺ T cells was tested ex vivo by studying the proliferative response of CD4⁺CD25⁻ T cells to donor and third-party cells in the presence and absence of CD4⁺CD25⁺ T cells with cells obtained in the posttransplant period. CD4⁺CD25⁻ T cells, but not CD4⁺CD25⁺ T cells, proliferated in response to donor and third-party antigen-presenting cells (APC). Co-culture of CD4⁺CD25⁻ with equal numbers of CD4⁺CD25⁺ T cells resulted in a reduction of 10% to 58% in the donor-stimulated proliferative response in all subjects tested (#1, #2, #3, #5, and #6). As shown in FIG. 7, the percent reduction in the third-party stimulated proliferative response in the presence of CD4⁺CD25⁺ T cells at a ratio of 1:1 was lower in 2 of the 3 studied recipients tested and higher in 1 (#5), as compared with the percent reduction in the anti-donor response in the presence of CD4⁺CD25⁺ cells at the same ratio.

The percentage of CD4⁺CD25⁺ T cells among CD4⁺ T cells was high in recipient #4 (32%) and #5 (31%) before hOKT3γ1 (Ala-Ala) administration, possibly suggesting an activated immune response despite the low frequency of CD69 T cells within the CD4⁺ compartment (#4, 5%; #5, 8%). In support of this interpretation are the markedly higher serum IL-2 levels in recipients #4 and #5 before and after hOKT3γ1 (Ala-Ala) administration as compared with the 4 insulin-independent recipients. The high serum IL-2 levels in recipients #4 and #5 may have reflected an ongoing inflammatory response, a high IL-2 producer genotype, or an altered IL-2/IL-2 receptor system; the result was a predisposition for resistance to induction of regulatory mechanisms, a predisposition that led to either rejection or autoimmune recurrence.

Furthermore, the exposure of recipient #5 to hOKT3γ1 (Ala-Ala) was substantially lower (Table 8), as compared with all 5 other recipients. This difference in hOKT3γ1 (Ala-Ala) exposure possibly also explains the lack of an inversion in this recipient's CD4⁺:CD8⁺ T cell ratio after anti-CD3 therapy, unlike in all 5 other recipients.

Autoantibodies. In recipients #2 and #6, GAD65 autoantibodies were positive pretransplant and remained positive after initiation of immunosuppression and after the islet transplant. GAD65 antibodies became positive posttransplant in recipients #1 and #5. ICA512 antibodies were positive pretransplant and remained positive after initiation of immunosuppression and after the islet transplant in recipient #2. None of the other recipients developed ICA512 antibodies posttransplant. Insulin autoantibodies were positive in 6 recipients pretransplant and in all but recipient #2 posttransplant.

Adverse events. One subject experienced a serious adverse event, a generalized rash later determined to be related to treatment with nystatin. Transient increases in liver transaminases (3 mild to moderate; 1 severe, AST >5 times the upper limit of normal for a single day), transient neutropenia (2 below 1000/μL and 1 below 500/μL); and lymphopenia were expected adverse events. Prolonged severe CD4⁺ T cell lymphocytopenia (consistent CD4⁺ T cell counts <200/μL was not observed. Mild to moderate adverse events, associated with the administration of the first 2 doses of hOKT3γ1 (Ala-Ala), included low grade fever, chills, and nausea and a transient erythematous rash. Other mild to moderate adverse events included aphthous mouth ulcers and weight loss. A change from normoalbuminuria to microalbuminuria was seen in 1 subject. A change from microalbuminuria to macroalbuminuria was observed in 1 subject. A change in LDL cholesterol from <100 to between 100 to 130 mg/dL was seen in 1 recipient. Procedural complications, serious infections, or serious, unexpected, and islet- or immunosuppression-related adverse events were not encountered.

In summary, the results from this example show that strategies are available to reverse diabetes with islets prepared from a single donor pancreas. Of six type 1 diabetes patients (C-peptide negative pretransplant with recurrent episodes of severe hypoglycemia), four achieved and sustained insulin independence, normoglycemia, and freedom from hypoglycemia after a single donor islet transplant. A fifth recipient showed partial islet graft function post-transplant. In the four insulin-independent recipients, HbA1c levels became and remained normal. These four showed a good acute C-peptide response to administration of intravenous arginine. Oral glucose tolerance tests post-transplant indicate tight glycemic control: three of the four insulin-independent patients showed an entirely normal response; and the fourth showed a response consistence with impaired glucose control based on American Diabetes Association criteria.

Superior metabolic outcome was observed as compared with studies in which islets from two to four donor pancreases were required to achieve insulin independence. Shapiro et al., New Engl. J. Med. 2000; 343:230-238; Ryan et al., Diabetes 2001; O50:710-719. In one of those studies of 12 insulin-independent recipients, four showed normal responses to oral glucose challenge; five, impaired; and three, diabetic.

In the two patients that did not achieve insulin independence (#4 and #5), the islets they received were prepared from donors with elevated vasopressor requirements (dopamine >20 μg/kg/min and/or norepinephrine at any dose) and with increased serum creatinine levels (1.8 mg/dL, #4; 5.2 mg/dL, #5) suggestive of relevant hypotension. Recipient #4 received an islet graft containing 0.32 units insulin/kg recipient body weight, and #5, 0.47. In contrast, the other four recipients received islet grafts containing 0.74 to 1.60 units insulin/kg recipient body weight. Aliquots from all four islet grafts that restored insulin independence also promptly reversed diabetes in diabetic nude mice within the first 4 days post-transplant. Aliquots from the two islet grafts that failed to restore insulin independence also failed to reverse diabetes in the mice in the first 4 weeks.

In addition to donor factors, the BMI (27 kg/m²), hemoglobin A1c (9.3%), and waist-to-hip ratio (0.96) of recipient #5 at the time of transplant were higher, as compared with the four insulin-independent recipients. Pre-transplant, the insulin requirement for recipient #5 was 0.8 units/kg higher than four of the five other recipients. Thus, recipient #5 showed clinical features of impaired insulin action, which may compromise the ability of an islet graft with marginal potency to restore insulin independence. Furthermore, the exposure of recipient #5 to hOKT3γ1 (Ala-Ala) was substantially lower, as compared with all five other recipients.

The percentage of CD4⁺CD25⁺ T cells among CD4⁻ T cells was high in recipients #4 (32%) and #5 (31%) before hOKT3γ1 (Ala-Ala) administration. Recipients #4 and #5 also had markedly higher serum IL-2 levels before and after hOKT3γ1 (Ala-Ala) administration.

### Example 5

### Summary of Islet Preparation Characteristics Preparations of human islet products (prepared using the method of Example 1 and transplanted to type 1 diabetic recipients) were characterized using the methods of Example 2. Table 10 provides a summary of the characteristics.

TABLE 10
Summary of Islet Preparation Characteristics

| Islet Product Test | N | Mean ± SD | Range |
|---|---|---|---|
| Liberase | | | |
| Wunsch units | 5 | 2,205 ± 103 | 2,082-2,363 |
| (collagenase activity) | | | |
| Caseinase units | 5 | 66,156 ± 11,338 | 56,437-85,651 |
| (proteolytic activity) | | | |
| Post Culture IE | 20 | 508,791 ± 171,576 | 253,960-875,583 |
| Post Culture IE/g pancreas | 20 | 5,350 ± 1,835 | 2,890-8,501 |
| IE/kg body weight | 20 | 7,881 ± 2,646 | 4,275-15,207 |
| Beta cells/kg body weight | 15 | 5.1 ± 3.4 | 0.9-11.0 |
| (×10⁶) | | | |
| Post Culture %-Recovery | 20 | 99 ± 14 | 66-131 |
| % Purity (DTZ) | 20 | 58.8 ± 8.8 | 40-70 |
| % Purity (Cell Comp) | 15 | 60.9 ± 10.3 | 48-82 |
| Tissue Volume Tx'd (mL) | 20 | 3.7 ± 2.6 | 1-10 |
| Low Glucose Stim | 20 | 0.162 ± 0.150 | 0.007-0.524 |
| (μU/ng/60 min)* | | | |
| High Glucose Stim | 20 | 0.481 ± 0.252 | 0.115-0.970 |
| (μU/ng/60 min)* | | | |
| Stimulation Index* | 20 | 6.0 ± 6.6 | 1.4-27.1 |
| Viability (%) - overnight* | 20 | 93.3 ± 4.4 | 86.6-99.9 |
| Viability (%) - 48 hour* | 20 | 96.8 ± 3.0 | 91.4-104.2 |
| OCR/DNA | 8 | 126 ± 47 | 75-204 |
| (nmol/min-mg)* | | | |
| ATP/DNA (pmol/μg)* | 6 | 123 ± 43 | 76-193 |
| Insulin Content (mU) | 20 | 64,825 ± 51,977 | 5,196-205,860 |
| DNA Content (μg) | 20 | 18,824 ± 17,891 | 2,525-55,111 |
| IE DNA Content (μg) - | 20 | 10,319 ± 9,707 | 1,649-35,001 |
| DTZ | | | |
| Insulin/DNA Ratio | 20 | 7.7 ± 8.7 | 0.4-27.6 |
| DNA/IE (ng) - DTZ | 20 | 19.1 ± 15.6 | 4.3-59.9 |
| Cell #/IE - DTZ | 20 | 2,734 ± 2,224 | 619-8,552 |
| Beta cells/IE - DTZ | 15 | 768 ± 996 | 146-3,923 |
| Beta Cell Number (×10⁶) | 15 | 315 ± 226 | 60-735 |
| Beta Cells (%) | 15 | 28.3 ± 7.3 | 17.8-45.9 |
| Alpha Cells (%) | 13 | 16.6 ± 9.1 | 6.3-33.3 |
| Delta Cells (%) | 13 | 10.7 ± 3.7 | 4.9-16.2 |
| PP Cells (%) | 13 | 5.5 ± 3.5 | 1.1-13.0 |
| Acinar Cells (%) | 15 | 24.1 ± 6.2 | 13.0-31.6 |
| Ductal Cells (%) | 15 | 9.5 ± 6.0 | 0.6-17.9 |
| Other Cells (%) | 13 | 6.3 ± 6.5 | 0.0-18.8 |
| Gram Stain - % negative | 20 | 100 | |
| Aerobic culture - | 20 | 100 | |
| % negative | | | |
| Anaerobic culture - | 20 | 100 | |
| % negative | | | |
| Fungal culture - % negative | 20 | 95 | |
| Mycoplasma - % negative | 20 | 100 | |
| Endotoxin | 20 | 1.5 ± 3.6 | 0.15-16.8 |
| (EU/kg body weight) | | | |
| Nude Mice (day of cure)* | | | |
| 750 IE | 3 | 6.3 ± 4.9 | 3-12 |
| 1000 IE | 5 | 3.2 ± 3.2 | 1-8 |
| 2000 IE | 10 | 2.8 ± 2.2 | 1-8 |

*Samples only taken from pure islet fraction.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising:
16.00 to 20.00 g/L raffinose;
4.00 to 6.00 g/L histidine;
4.00 to 5.00 g/L sodium hydroxide;
30.00 to 40.00 g/L lactobionic acid;
0.30 to 0.50 g/L potassium hydroxide;
0.05 to 0.10 g/L calcium chloride;
1.00 to 1.50 g/L magnesium sulfate;
3.00 to 4.00 g/L sodium phosphate monobasic; and
19.00 to 21.00 g/L pentastarch.

2. The composition of claim 1, said composition further comprising 8.00 to 12.00 U/mL heparin; and 8.00 to 12.00 μg/mL insulin.

3. The composition of claim 2, said composition further comprising a population of isogenic human pancreatic islets.

4-5. (canceled)

6. The composition of claim 2, said composition further comprising iodixanol.

7. The composition of claim 6, said composition further comprising a population of isogenic human pancreatic islets.

8-9. (canceled)

10. A composition comprising
5.00 to 6.00 g/L mannitol;
0.50 to 0.70 g/L sodium hydroxide;
5.00 to 7.00 g/L sodium chloride;
0.25 to 0.40 g/L potassium hydroxide;
0.05 to 0.15 g/L calcium chloride;
0.15 to 0.25 g/L magnesium sulfate; and
3.00 to 4.00 g/L sodium phosphate monobasic.

11. The composition of claim 10, said composition further comprising 8.00 to 12.00 U/mL heparin.

12. The composition of claim 10, said composition further comprising 1,000 to 3600 Wunsch units of collagenase.

13. The composition of claim 11, said composition further comprising 1,000 to 3600 Wunsch units of collagenase.

14. The composition of claim 10, said composition further comprising a trypsin inhibitor.

15. The composition of claim 13, said composition further comprising a trypsin inhibitor.

16-18. (canceled)

19. The composition of claim 13, said composition further comprising a population of isogenic human pancreatic islets.

20-21. (canceled)

22. A composition comprising:
16.00 to 20.00 g/L raffinose;
4.00 to 6.00 g/L histidine;
4.00 to 5.00 g/L sodium hydroxide;
30.00 to 40.00 g/L lactobionic acid;
0.30 to 0.50 g/L potassium hydroxide;
0.05 to 0.10 g/L calcium chloride;
1.00 to 1.50 g/L magnesium sulfate;
3.00 to 4.00 g/L sodium phosphate monobasic;
15.00 to 25.00 g/L pentastarch; and
200 to 300 ml/L iodixanol.

23. The composition of claim 22, said composition further comprising a population of isogenic human pancreatic islets.

24-25. (canceled)

26. A chemically defined culture medium comprising insulin, zinc sulfate, selenium, and transferrin, wherein said medium is effective for maintaining viability of human pancreatic islets under culture conditions.

27. The culture medium of claim 26, further comprising sodium pyruvate, HEPES (N-[2-Hydroxyethyl]piperazine-N′[2-ethanesulfonic acid]), and human serum albumin (HSA).

28. The culture medium of claim 27, further comprising 8.00 to 12.00 U/mL heparin.

29. The culture medium of claim 28, said composition further comprising a population of isogenic human pancreatic islets.

30-31. (canceled)

32. A composition comprising
8.00 to 10.00 g/L mannitol;
3.00 to 6.00 g/L L-histidine;
18.00 to 21.00 g/L gluconic acid;
0.50 to 2.00 g/L potassium hydroxide;
0.01 to 0.05 g/L calcium chloride;
0.50 to 2.00 g/L magnesium sulfate;
0.40 to 0.70 g/L nicotinamide;
0.30 to 0.70 g/L pyruvate; and
1.50 to 3.50 g/L potassium phosphate monobasic.

33. The composition of claim 32, said composition further comprising a population of isogenic human pancreatic islets.

34-35. (canceled)
